SEQUENCE LISTING (14 sequences)

SEQ ID NO: 1 - DNA, 2347 bp, Homo sapiens
  CDS (25)..(1617)
gacccccggtctcgcccccgaaacatgactcgcgatttcaaacctggagacctcatcttcgccaagatgaaaggttatccccattggccagctcgagtagacgaagttcctgatggagctgtaaagccacccacaaacaaactacccattttcttttttggaactcatgagactgcttttttaggaccaaaggatatatttccttactcagaaaataaggaaaagtatggcaaaccaaataaaagaaaaggttttaatgaaggtttatgggagatagataacaatccaaaagtgaaattttcaagtcaacaggcagcaactaaacaatcaaatgcatcatctgatgttgaagttgaagaaaaggaaactagtgtttcaaaggaagataccgaccatgaagaaaaagccagcaatgaggatgtgactaaagcagttgacataactactccaaaagctgccagaagggggagaaagagaaaggcagaaaaacaagtagaaactgaggaggcaggagtagtgacaacagcaacagcatctgttaatctaaaagtgagtcctaaaagaggacgacctgcagctacagaagtcaagattccaaaaccaagaggcagacccaaaatggtaaaacagccctgtccttcagagagtgacatcattactgaagaggacaaaagtaagaaaaaggggcaagagggaaaacaacctaaaaagcagcctaagaaggatgaagagggccagaaggaagaagataagccaagaaaagagccggataaaaaagaggggaagaaagaagttgaatcaaaaaggaaaaatttagctaaaacaggggttacttcaacctccgattctgaagaagaaggagatgatcaagaaggtgaaaagaagagaaaaggtgggaggaactttcagactgctcacagaaggaatatgctgaaaggccaacatgagaaagaagcagcagatcgaaaacgcaagcaagaggaacaaatggaaactgagcagcagaataaagatgaaggaaagaagccagaagttaagaaagtggagaagaagcgagaaacatcaatggattctcgacttcaaaggatacatgctgagattaaaaattcactcaaaattgataatcttgatgtgaacagatgcattgaggccttggatgaacttgcttcacttcaggtcacaatgcaacaagctcagaaacacacagagatgattactacactgaaaaaaatacggcgattcaaagttagtcaggtaatcatggaaaagtctacaatgttgtttaacaagtttaagaacatgttcttggttggtgaaggagattccgtgatcacccaagtgctgaataaatctcttgctgaacaaagacagcatgaggaagcgaataaaaccaaagatcaagggaagaaagggccaaacaaaaagctagagaaggaacaaacagggtcaaagactctaaatggaggatctgatgctcaagatggtaatcagccacaacataacggggagagcaatgaagacagcaaagacaaccatgaagccagcacgaagaaaaagccatccagtgaagagagagagactgaaatatctctgaaggattctacactagataactaggttgacatacctggaatatagagaacacttgagaagtttgtaatggttttcatttgaaatagactgctgaaagttttaaatttttataagcataggtttgatgttgaaaacttgttttgagggagaaaatccctttgttttaaagtaaagtaaacattatcgctaagtgtacttgtgcagtattaacagctacattatacagtaaatgtgggatggaatccatttaggaaatgttaaactgcttttccagacatggttgtagcatattttcaattagtgtgtgtatgttaatgtgtaattgatagtagaacaaagttacatttttaaaactgctacttgtataaaccttgcctcttttcccaaatactgtgggttttgtgcatagtttttacaaaccttggatttaccagactgtcttttcactgtttgtgggttttgtagaagttacacatttttatggtagataaaatgttacttctatacaagtactcactccctttttatcaaaagttaattttaatctcacagtctacattgtgctacattatccagcttctttggaacaatgtgtgctctgtatggttttttttggtatgacaactaattaagcaactgacattgaactgagaattctacaaactataaaacattaatttttgaaggtaatttagttttgtggctgggcattcagtgaagtcttaggacttctttgcagacaactgactgggta

SEQ ID NO: 2 - protein, 530 aa, Homo sapiens
MTRDFKPGDLIFAKMKGYPHWPARVDEVPDGAVKPPTNKLPIFFFGTHETAFLGPKDIFPYSENKEKYGKPNKRKGFNEGLWEIDNNPKVKFSSQQAATKQSNASSDVEVEEKETSVSKEDTDHEEKASNEDVTKAVDITTPKAARRGRKRKAEKQVETEEAGVVTTATASVNLKVSPKRGRPAATEVKIPKPRGRPKMVKQPCPSESDIITEEDKSKKKGQEGKQPKKQPKKDEEGQKEEDKPRKEPDKKEGKKEVESKRKNLAKTGVTSTSDSEEEGDDQEGEKKRKGGRNFQTAHRRNMLKGQHEKEAADRKRKQEEQMETEQQNKDEGKKPEVKKVEKKRETSMDSRLQRIHAEIKNSLKIDNLDVNRCIEALDELASLQVTMQQAQKHTEMITTLKKIRRFKVSQVIMEKSTMLFNKFKNMFLVGEGDSVITQVLNKSLAEQRQHEEANKTKDQGKKGPNKKLEKEQTGSKTLNGGSDAQDGNQPQHNGESNEDSKDNHEASTKKKPSSEERETEISLKDSTLDN

SEQ ID NO: 3 - DNA, 1763 bp, Homo sapiens
  CDS (78)..(1079)
gaattcgcggccgccccgcgccgccgcatctcctcgccgcctcccgggcttcggacccccggtctcgcccccgaaacatgactcgcgatttcaaacctggagacctcatcttcgccaagatgaaaggttatccccattggccagctcgagtagacgaagttcctgatggagctgtaaagccacccacaaacaaactacccattttcttttttggaactcatgagactgcttttttaggaccaaaggatatatttccttactcagaaaataaggaaaagtatggcaaaccaaataaaagaaaaggttttaatgaaggtttatgggagatagataacaatccaaaagtgaaattttcaagtcaacaggcagcaactaaacaatcaaatgcatcatctgatgttgaagttgaagaaaaggaaactagtgtttcaaaggaagataccgaccatgaagaaaaagccagcaatgaggatgtgactaaagcagttgacataactactccaaaagctgccagaagggggagaaagagaaaggcagaaaaacaagtagaaactgaggaggcaggagtagtgacaacagcaacagcatctgttaatctaaaagtgagtcctaaaagaggacgacctgcagctacagaagtcaagattccaaaaccaagaggcagacccaaaatggtaaaacagccctgtccttcagagagtgacatcattactgaagaggacaaaagtaagaaaaaggggcaagagggaaaacaacctaaaaagcagcctaagaaggatgaagagggccagaaggaagaagataagccaagaaaagagccggataaaaaagaggggaagaaagaagttgaatcaaaaaggaaaaatttagctaaaacaggggttacttcaacctccgattctgaagaagaaggagatgatcaagaaggtgaaaagaagagaaaaggtgggaggaactttcagactgctcacagaaggaatatgctgaaaggccaacatgagaaagaagcagcagatcgaaaacgcaagcaagaggaacaaatggaaactgagcaccaaacaacatgtaatctacagtaataaaaaatatatctcattttgggctcaaagcattaatccagttactgaaaagagaatacaagtggagcaaacaagagatgaagatcttgatacagactcattggactgaatttcccccttccccccatgatggaagaatgttcagattctaaattgaggacttcattattaatggcattactgtgttatgattaacaaatttcttgtaaggtacacactacatactaaggtcggccatcattccgtttttttttttttttttttttttaaccaagcttaaaatgaagcttaaaatgaagctttgtgtttgaaagtaataacaagctcagacgaagatggtggttgtacattattcatctagaaaatataaaaattcattttgttttgaagctagttattaaactggaatagcagttatatccctgagaatggggcccttctcttgacattccttttgttgtttaattctttagaatcttaataaatgtttttttaatcctgagagattaaacagtagtagacttgttaagaatgaaactgtaaccaaaattttaaaataaagttttttttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagcggccgcgaattc

SEQ ID NO: 4 - protein, 333 aa, Homo sapiens
MTRDFKPGDLIFAKMKGYPHWPARVDEVPDGAVKPPTNKLPIFFFGTHETAFLGPKDIFPYSENKEKYGKPNKRKGFNEGLWEIDNNPKVKFSSQQAATKQSNASSDVEVEEKETSVSKEDTDHEEKASNEDVTKAVDITTPKAARRGRKRKAEKQVETEEAGVVTTATASVNLKVSPKRGRPAATEVKIPKPRGRPKMVKQPCPSESDIITEEDKSKKKGQEGKQPKKQPKKDEEGQKEEDKPRKEPDKKEGKKEVESKRKNLAKTGVTSTSDSEEEGDDQEGEKKRKGGRNFQTAHRRNMLKGQHEKEAADRKRKQEEQMETEHQTTCNLQ

SEQ ID NO: 5 - protein, 325 aa, Homo sapiens
MTRDFKPGDLIFAKMKGYPHWPARVDEVPDGAVKPPTNKLPIFFFGTHETAFLGPKDIFPYSENKEKYGKPNKRKGFNEGLWEIDNNPKVKFSSQQAATKQSNASSDVEVEEKETSVSKEDTDHEEKASNEDVTKAVDITTPKAARRGRKRKAEKQVETEEAGVVTTATASVNLKVSPKRGRPAATEVKIPKPRGRPKMVKQPCPSESDIITEEDKSKKKGQEGKQPKKQPKKDEEGQKEEDKPRKEPDKKEGKKEVESKRKNLAKTGVTSTSDSEEEGDDQEGEKKRKGGRNFQTAHRRNSLKGQHEKEAADRKEKQEEQMETE

SEQ ID NO: 6 - protein, 8 aa, Homo sapiens
HQTTCNLQ

SEQ ID NO: 7 - DNA, 47 bp, Artificial Sequence
  Description of Artificial Sequence oligonucleotide
gatttcaarccnggngatctntttgcnaaratgaarggntacccnca

SEQ ID NO: 8 - protein, 179 aa, Homo sapiens
MTRDFKPGDLIFAKMKGYPHWPARVDEVPDGAVKPPTNKLPIFFFGTHETAFLGPKDIFPYSENKEKYGKPNKRKGFNEGLWEIDNNPKVKFSSQQAATKQSNASSDVEVEEKETSVSKEDTDHEEKASNEDVTKAVDITTPKAARRGRKRKAEKQVETEEAGVVTTATASVNLKVSPK

SEQ ID NO: 9 - DNA, 1001 bp, Homo sapiens
gacccccggtctcgcccccgaaacatgactcgcgatttcaaacctggagacctcatcttcgccaagatgaaaggttatccccattggccagctcgagtagacgaagttcctgatggagctgtaaagccacccacaaacaaactacccattttcttttttggaactcatgagactgcttttttaggaccaaaggatatatttccttactcagaaaataaggaaaagtatggcaaaccaaataaaagaaaaggttttaatgaaggtttatgggagatagataacaatccaaaagtgaaattttcaagtcaacaggcagcaactaaacaatcaaatgcatcatctgatgttgaagttgaagaaaaggaaactagtgtttcaaaggaagataccgaccatgaagaaaaagccagcaatgaggatgtgactaaagcagttgacataactactccaaaagctgccagaagggggagaaagagaaaggcagaaaaacaagtagaaactgaggaggcaggagtagtgacaacagcaacagcatctgttaatctaaaagtgagtcctaaaagaggacgacctgcagctacagaagtcaagattccaaaaccaagaggcagacccaaaatggtaaaacagccctgtccttcagagagtgacatcattactgaagaggacaaaagtaagaaaaaggggcaagagggaaaacaacctaaaaagcagcctaagaaggatgaagagggccagaaggaagaagataagccaagaaaagagccggataaaaaagaggggaagaaagaagttgaatcaaaaaggaaaaatttagctaaaacaggggttacttcaacctccgattctgaagaagaaggagatgatcaagaaggtgaaaagaagagaaaaggtgggaggaactttcagactgctcacagaaggaatatgctgaaaggccaacatgagaaagaagcagcagatcgaaaacgcaagcaagaggaacaaatggaaactgagca

SEQ ID NO: 10 - DNA, 24 bp, Homo sapiens
  CDS (1)..(24)
caccaaacaacatgtaatctacag

SEQ ID NO: 11 - protein, 25 aa, Homo sapiens
  UNSURE (1)..(25): Xaa represents any amino acid residue
XXDFKPGDLIFAKMKGYPHXPAXVD

SEQ ID NO: 12 - protein, 23 aa, Homo sapiens
  UNSURE (1): Xaa or Gly or Lys
XYPXSPAXVDEVPDXAVKPPTNK

SEQ ID NO: 13 - protein, 14 aa, Homo sapiens
GFNEGLWEIDNNPK

SEQ ID NO: 14 - protein, 205 aa, Homo sapiens
QQNKDEGKKPEVKKVEKKRETSMDSRLQRIHAEIKNSLKIDNLDVNRCIEALDELASLQVTMQQAQKHTEMITTLKKIRRFKVSQVIMEKSTMLFNKFKNMFLVGEGDSVITQVLNKSLAEQRQHEEANKTKDQGKKGPNKKLEKEQTGSKTLNGGSDAQDGNQPQHNGESNEDSKDNHEASTKKKPSSEERETEISLKDSTLDN

(19) United States
(12) Patent Application Publication
(10) Pub. No.: US 2005/0042631 A1
(43) Pub. Date: Feb. 24, 2005

(54) Cloning and characterization of two novel m-RNA transcription factors

(75) Inventor: Hui Ge, Gaithersburg, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(73) Assignee: Government of The U.S.A. as Represented by the Secretary of the Dept. of Health & Human Services

(21) Appl. No.: 10/819,860

(22) Filed: Apr. 6, 2004

Related U.S. Application Data
(62) Division of application No. 09/830,942, filed on May 2, 2001, now abandoned.
(60) Provisional application No. 60/108,248, filed on Nov. 13, 1998.

(51) Int. Cl. C12Q 1/68; C07H 21/04; C12N 9/22

(52) U.S. Cl. 435/6; 435/69.1; 435/199; 435/320.1; 435/325; 536/23.2

(57) ABSTRACT

DNA and protein sequences are disclosed for coactivators of mRNA transcription identified as p52 and p75. The p52 sequence also enhances ASF/SF2-mediated pre-mRNA splicing activity. The disclosure also includes specific binding agents (such as antibodies) that recognize these activators, methods of enhancing transcription using the activators, methods of treating disease caused by mutations, therapeutic compositions that include the activators, recombinant DNA molecules, probes, and transformed cells that incorporate the DNA sequence to express p52 and p75. The disclosure also includes methods of diagnosis and treatment of diseases caused by an underexpression of p52 and/or p75, including tumors such as breast adenocarcinomas.

### FIELD

This invention relates to nucleic acid and amino acid sequences corresponding to p52 and p75 that are active in cotranscriptional activation and alternative splicing of mRNA, and are underexpressed in certain cancers, such as breast cancers.

### BACKGROUND

In eukaryotes, RNA molecules are transcribed from a DNA template by one of three RNA polymerases. Only RNA polymerase II (pol II) transcribes the genes whose RNAs will be translated into proteins. The pre-messenger RNA (pre-mRNA) transcript contains exon and intron sequences. The introns are removed from the transcript, by a process called splicing, producing an mRNA molecule that codes directly for a protein.

Proper pol II transcription has emerged as a predominant mechanism linked to development, differentiation, metabolism and human disease. Modulation of transcriptional activation by RNA polymerase II is a complex multistep process controlled by at least three distinct classes of transcription factors. The first class includes the general transcription factors, TFIIA, TFIIB, TFIID. TFIIE, TFIIF and TFIIH, in addition to RNA polymerase II, and mediates accurate transcription initiation through common core promoter elements (for reviews see Roeder, Trends Biochem. Sci. 21:327-35, 1996; Orphanides et al., Genes Dev. 10:2657-83, 1996). The second class consists of gene-specific regulators that bind to DNA elements distal to core promoter elements and regulate the rate of transcription by the general transcription apparatus.

The third class is a diverse and more recently identified group of cofactors, including both coactivators and corepressors, that are essential for, or modulate, functional interactions between DNA-bound gene specific regulators and the general transcription factors. Members of this group include gene-specific cofactors associated with DNA-binding regulatory factors, cofactors associated with the basal transcriptional machinery and various soluble cofactors (Kaiser and Meisterernst. Trends Biochem. Sci. 21:342-5, 1996). Therefore, transcriptional activation of class II genes involves a complex interplay of protein-DNA and protein-protein interactions.

An apparently distinct set of general coactivators are positive cofactors (PCs), which have been identified in human HeLa cells. At least four PCs (PC1, PC2, PC3 and PC4) have been separated and completely or partially purified from the upstream stimulatory activity (USA) fraction, while two less well characterized PCs (PC5 and PC6) have been found in other HeLa cell nuclear extract-derived fractions (Kaiser and Meisterernst, Trends Biochem. Sci. 21:342-5, 1996). The best characterized PC is PC4, which is a single- and double-stranded DNA binding protein that mediates activator-dependent transcription, in a TATA box binding protein (TBP) and TBP-associated factors (TAF)-dependent manner, but is not required for basal activity in an in vitro reconstituted transcription system. PC4 acts as a general transcriptional coactivator for a variety of activators and, consistent with its role as an adapter, directly interacts both with activation domains of regulatory factors and with the general transcription factor TFIIA. All these activities of PC4 are negatively regulated in vivo by phosphorylation.

Once synthesis of pre-mRNA is initiated in the eukaryotic nucleus, the introns must be accurately removed through splicing from pre-mRNA and the 3′ end must be processed through cleavage/polyadenylation to generate mature mRNAs. In addition to conserved sequence elements, including 5′ and 3′ splice sites and branch points, several small nuclear ribonucleoprotein particles (snRNPs) are essential for the spliceosome assembly (reviewed by Steitz et al., Functions of the abundant U-snRNPs. In Structure and function of major and minor small nuclear ribonucleoprotein particles. M. Birnsteil, ed (New York: Springer), pp. 115-54, 1988; Moore et al. Splicing of precursors to messenger RNAs by the spliceosome. In The RNA World, R. F. Gesteland and J. F. Atkins, eds. (Cold Spring Harbor, N.Y.: Cold Spring Harbor Laboratory Press), pp. 303-58, 1993; Madhani and Guthrie, Annu. Rev. Genet. 28:1-26, 1994; Sharp, Cell 77:805-15, 1994). Despite the complexity of alternative splicing pathways, which has hampered studies on splice site selection, some progress has been made in the identification and characterization of the serine-arginine rich (SR) protein family, which is a group of non-snRNP splicing factors that play important roles in both constitutive and alternative splicing by recognizing splicing enhancers and interacting with other splicing factors (reviewed by Maniatis, Science 251:33-4, 1991; Horowitz and Krainer, Trends Genet. Sci. 10:100-6, 1994; Fu, RNA 1:663-80, 1995; Manley and Tacke, Genes Dev. 10: 1569-79, 1996; Valcarcel and Green, Trends Biochem. Sci. 21:296-301, 1996). The alternative/essential splicing factor ASF/SF2 was the first SR protein discovered in a mammalian system based on its function in alternative and constitutive splicing assays (Ge and Manley, Cell 62:25-34, 1990; Ge et al., Cell 66:373-82, 1991; Krainer et al., Genes Dev. 4:1158-71, 1990; Krainer et al., Cell 66:383-94, 1991).

Pre-mRNA splicing and other processing events can occur in cell-free systems (nuclear extracts) using pre-made precursor RNAs as substrates, but there is accumulating evidence that the transcription of class II genes and pre-mRNA processing are coupled in vivo. More than a decade ago, it was found that snRNPs and other splicing components were co-localized at transcriptionally active chromosomal sites (Sass and Pederson, J. Mol. Biol. 180:911-26, 1984; Fakan et al., J. Cell Biol. 103:1153-7, 1986) and that intron removal could occur prior to transcription termination (Beyer and Osheim, Genes Dev. 2:754-65, 1988). More recently, studies have revealed the co-localization of viral or cellular pre-mRNA and/or splicing factors with the RNA polymerase II transcription machinery in the nuclear sub-compartments known as speckles, further supporting the existence of coordination between transcription and pre-mRNA splicing (Huang and Spector, Genes Dev. 5:2288-2302, 1991; Kim et al., Genes Dev. 6:2569-79, 1992; Xing et al. Science 259:1326-30, 1993: Jimenez-Garcia and Spector, Cell 73:47-59, 1993). Nevertheless splicing does not invariably take place at these sites (Mattaj, Nature 372:727-8, 1994. Zhang et al., Nature 372:809-12, 1994; Zeng et al., EMBO J. 16:1401-12, 1997). Bauran and Wieslander (Cell 76:183-92, 1994) found that introns of the Balbiani Ring 1 pre-mRNA were excised during pre-mRNA synthesis. By using transient and stable transfection assays, Huang and Spector (J. Cell Biol. 133:719-32, 1996) found that splicing factors could be recruited to the sites of active transcription for intron-containing templates, but not for intron-less templates, inviting speculation that transcription and splicing of pre-mRNA are linked. In spite of these advances, biochemical insight is lacking into how splicing factors are recruited to the nascent transcripts.

### SUMMARY

The present invention takes advantage of the discovery of two proteins, p52 and p75, which are coactivators of mRNA transcription. In addition, the p52-protein has been found to enhance ASF/SF2-mediated pre-mRNA splicing. The sequences of these proteins have been determined, as have DNA sequences encoding them. The levels of both RNA and protein expression of p52 and p75 in certain cancer cells is dramatically decreased.

The present invention therefore includes a purified polypeptide having the amino acid sequence of p52, p75, or subsequences thereof, shown in the accompanying Sequence Listings, as well as nucleic acid sequences encoding the polypeptides. Alternatively, the purified polypeptide has an activity of p52 or p75. When the purified polypeptide has the activity of p52, it acts as a general coactivator of transcription, and selectively interacts with ASF/SF2 to elevate proximal t 5′ splice site selection of SV40 early pre-mRNA in the presence of HeLa cell nuclear extract, and activates splicing in the presence of HeLa cell S100 extract and ASF/SF2. The p52 polypeptide may also enhance transcription of transcriptional activators containing an acidic activation domain, a proline-rich activation domain, or a glutamine-rich activation domain.

In some embodiments, the purified polypeptide has cotranscriptional activator activity, and includes an amino acid sequence selected from the group of the amino acid sequence shown in SEQ ID NO 2, amino acid sequences that differ from those specified in SEQ ID NO 2 by one or more conservative amino acid substitutions, and amino acid sequences having at least 75% sequence identity to such sequences, but which retain the cotranscriptional activator activity of the amino acid sequence encoded by SEQ ID NO 2. The purified polypeptide can also include the amino acid sequence shown in SEQ ID NO 4, amino acid sequences that differ from that specified in SEQ ID NO 4 by one or more conservative amino acid substitutions, and amino acid sequences having at least 75% sequence identity to such sequences, but which retain the cotranscriptional activator activity of the amino acid sequence of SEQ ID NO 4, and/or the ASF/SF2-mediated pre-mRNA splicing activity of the amino acid sequence shown in SEQ ID NO 4.

Also included in the invention are isolated polynucleotides encoding such proteins, or a polynucleotide capable of hybridizing to such polynucleotides under stringent conditions, and which encodes a protein that retains the cotranscriptional activator activity of p52 or p75. In some embodiments, in which the encoded protein has the activity of p52, the polynucleotide may also have the ASF/SF2-mediated pre-mRNA splicing activity of p52.

In some embodiments, the invention also includes an antibody generated against the polypeptides of the invention, methods of enhancing transcription in a mammalian cell by exposing that cell to an amount of the polypeptide sufficient to enhance transcription, and methods of enhancing ASF/SF2-mediated pre-mRNA splicing in a mammalian cell by contacting that cell with a sufficient amount of the polypeptide defined in claim 21. The methods can also include treating a disease caused by defects in transcription by administering a therapeutic amount of a polypeptide such as p52 or p75, or a variant thereof. Alternatively, the disease may be caused by defects in ASF/SF2-mediated pre-mRNA splicing, and the treatment can be administration of a therapeutic amount of p52, or a variant thereof. The therapeutically effective amount of the polypeptide can be administered in combination with a pharmaceutically acceptable carrier.

Also included are processes of diagnosing a disease, or a susceptibility to a disease, related to abnormal expression of the p52 or p75 protein, such as under-expression, by identifying a mutation in a nucleic acid sequence encoding the protein in a sample derived from a patient. Alternatively, variant proteins which are associated with such diseases can be detected in a subject. In yet another method, diagnosing a disease or a susceptibility to a disease related to an under-expression of the polypeptide of SEQ ID NO 5 involves quantitating the level of the polypeptide of SEQ ID NO 5 in a sample derived from a patient. In particular embodiments, the disease diagnosed is cancer, such as adenocarcinoma of the breast.

Also provided in the present invention is a method of treating a disease caused by a mutation in the polynucleotide of p52 or p75 by supplying therapeutically effective amounts of a polypeptide product or the polynucleotide.

Other embodiments of the invention may include a recombinant nucleic acid molecule in which a promoter sequence is operably linked to a nucleic acid sequence encoding a protein having the activity of p52 or p75, cells transformed with the recombinant nucleic acid molecule, or a transgenic animal into which the recombinant nucleic acid molecule has been introduced.

Another embodiment of the present invention are cells in which p52 and/or p75 is functionally deleted. In a specific embodiment, the cells are DT40 cells.

Yet other embodiments of the invention include probes and primers, for example an oligonucleotide that is at least 20, 30 or 50 contiguous nucleotides of the sequences shown in SEQ ID NO 9; or at least 6, 7 or 8 contiguous nucleotides of SEQ ID NO 10. The polynucleotides of the invention may also be isolated nucleic acid molecules that hybridizes with a nucleic acid molecule that includes the sequence shown in SEQ ID Nos.: 1 or 3, under wash conditions of 65° C. 0.2×SSC and 0.1% SDS; and which encodes a protein having p52 or p75 protein biological activity.

The foregoing and other objects, features, and advantages of the invention will become more apparent from the following detailed description of a preferred embodiment which proceeds with reference to the accompanying drawings.

### BRIEF DESCRIPTION OF THE DRAWINGS

FIG. 1 shows the p52 cDNA sequence, with the protein coding region underlined; the start codon (ATG) and stop codon (TAA) are in bold.

FIG. 2 shows the p75 cDNA sequence with the protein coding region underlined. The start and stop codons are in bold, and sequences identical to p52 are capitalized.

FIG. 3 shows the amino acid sequences for p52 (A) and p75 (B). The amino acid sequences obtained from microsequencing of N-terminal (residues 4-26) and internal (residues 17-39 and 76-89) peptides are underlined. The highly charged C-terminal domain of p52 is shaded. The boxed amino acid residues indicated in (B) is the C-terminal region unique to p75.

FIG. 4 is a schematic diagram showing a comparison of p52 and p75 amino acid sequences, with regions of homology noted.

FIG. 5 shows the Northern analysis of p52 (A) and p75 (B) RNA expression. A schematic representation of p52 and p75 protein structures and probes used are shown in (C).

FIG. 6 shows the SDS-PAGE analysis of recombinant p52 and p75 expression. Proteins were visualized by Coomassie blue staining (left panel) or immunoblot using polyclonal anti-p52 antibodies (right panel) which recognize both p52 and p75.

FIG. 7 shows the results of an in vitro transcription assay. Recombinant p52, p75 and PC4 were incubated with purified factors either in the presence (+) or in the absence (−) of activator GAL4-AH as indicated. Transcripts of pG₅HMC2AT (activated template) and pMLΔ53 (control basal template) are indicated by arrows.

FIG. 8 shows the results of an in vitro transcription assay in the presence of different activators. (A) Transcription of either GAL4-VP16 (lanes 1-10) or GALA-IE (lanes 11-20). (B) Transcription of GAL4-CTF (lanes 2, 6, 10 and 14), GAL4-Sp1 (lanes 3, 7, 11 and 15), GAL4-EIA (lanes 5, 9, 13 and 17), and GALA-IE (lanes 4, 8, 12 and 16). (C) Quantitative representation of B. The relative transcription activity from lane 1 (absence of activator and coactivator) was normalized as 1.

FIG. 9 shows the results of a protein binding assay between p52 and p75 and the VP16 activation domain. (A) Coomassie blue staining of purified GST fusion proteins. (B) ³²P-labeled recombinant p52 (lanes 14) or p75 (lanes 5-8) bind to the VP16 activation domain fusion protein.

FIG. 10 is a digital image showing the result of slot blot analysis of the interaction of p52 and p75 with various transcription factors.

FIG. 11 shows the results of a protein binding assay examining the interaction of p52 with PC4 and ASF/SF2. (A) Farwestern blot of HeLa cell nuclear extract hybridized with either ³²P-labeled GST-K-p52 (left panel) or GST-K (control, right panel). (B) shows the direct specific interaction of p52 with native ASF/SF2. (C) Six histidine-tagged recombinant ASF/SF2 (lane 1), GST-fused wild type ASF/SF2 (GST-ASF, lane 2), GST-fused RNA-binding domains of ASF/SF2 (GST-ARS, lane 3) and GST-fused RS domain of ASF/SF2 (GST-RS, lane 4) were probed with ³²P-labeled GST-K-p52. (D) Schematic representation of recombinant ASF/SF2 proteins.

FIG. 12 shows shows the results of an Sp1-dependent in vitro transcription assay. (A) in vitro reconstituted transcription assays. (B) Quantitative representation of A.

FIG. 13 shows shows the results of an in vitro splicing assay using HeLa cell nuclear extract. (A) in vitro splicing assays with spliced products and intermediates shown schematically on the right. (B) Schematic diagram representing the SV40 early pre-mRNA derived from plasmid pSV166.

FIG. 14 shows the results of an in vitro splicing assays using HeLa cell S100 extract (cytoplasmic fraction) in the presence (+) or absence (−) of added ASF/SF2. Precursor RNA, spliced products and intermediates are indicated schematically at right.

FIG. 15 is a digital image of a (A) Northern blot and a (B) Western blot showing the level of p52 and p75 (A) RNA and (B) protein expression in several cancer cell lines.

### Sequence Listing

The nucleic and amino acid sequences listed in the accompanying sequence listing are shown using standard letter abbreviations for nucleotide bases, and three letter code for amino acids. Only one strand of each nucleic acid sequence is shown, but the complementary strand is understood as included by any reference to the displayed strand.

SEQ ID NO 1 shows the nucleotide sequence of human p75, GenBank Accession No. AF098483.

SEQ ID NO 2 shows the amino acid sequence of human p75 positions 1-530, GenBank Accession No. AAC97946.

SEQ ID NO 3. shows the nucleotide sequence of human p52, GenBank Accession No. AF098482.

SEQ ID NO 4 shows the amino acid sequence of human p52 positions 1-333, GenBank Accession No. AAC97945.

SEQ ID NO 5 shows the amino acid sequence of human p52 positions 1-325.

SEQ ID NO 6 shows the amino acid sequence of human p52 positions 326-333.

SEQ ID NO 7 shows the nucleotide sequence of an oligonucleotide used to screen a cDNA library.

SEQ ID NO 8 shows the N-terminal amino acid sequence of both human p52 and human p75, positions 1-179.

SEQ ID NO 9 shows the nucleotide sequence of the 5′ region of both p52 and p75.

SEQ ID NO 10 shows the nucleotide sequence which corresponds to amino acid residues 326-333 of human p52.

SEQ ID NOs 11-13 show the amino acid sequences for the peptide fragments resulting from the N-terminal sequencing of a 75 kDa polypeptide.

SEQ ID NO 14 shows the amino acid sequence of human p75 positions 326-530.

### DETAILED DESCRIPTION OF SEVERAL EMBODIMENTS

### Abbreviations and Definitions

The following abbreviations and definitions are used herein:
BSA bovine serum albumin
DTT dithiotheitol
FPLC fast performance liquid chromatography
GST glutathione-S-transferase
HMK heart muscle kinase
IPTG isopropyl P-D-thiogalactopyranoside
PBS phosphate buffered saline
PCs positive cofactors
PMSF phenylmethylsulfonyl fluoride
RT room temperature
SDS-PAGE sodium dodecyl sulfate-polyacrylamide gel electrophoresis
USA Upstream stimulatory activity
UTR untranslated region

293 cells: A cell line derived from a human embryonic kidney which has been transformed with adenovirus 5 DNA.

HeLa cells: A.T.C.C. (Manassas, Va.) number CCL-2. A human cell line derived from an adenocarcinoma of the cervix. 3-10 HeLa cells: HeLa cells that stably express recombinant full-length TBP (TATA box binding protein).

COS-7 cells: A.T.C.C. (Manassas, Va.) number CRL-1651. African Green Monkey kidney cells transformed with SV40.

MCF 7 cells: A.T.C.C. (Manassas, Va.) number HTB-22. A cell line derived from a human adenocarcinoma of the mammary gland with pleural effusion.

MDA-MB-231: A.T.C.C. (Manassas, Va.) number HTB-26. A cell line derived from a human adenocarcinoma of the mammary gland with pleural effusion.

MDA-MB468: A.T.C.C. (Manassas, Va.) number HTB-132. A cell line derived from a human adenocarcinoma of the mammary gland.

Adenovirus E1A: a transcriptional activator containing an acidic activation domain.

Animal: Living multicellular vertebrate organisms, a category which includes, for example, mammals and birds.

ASF/SF2: Alternative splicing factor/splicing factor 2. This splicing factor is a member of the serine-arginine rich (SR) protein family. When added to HeLa cell nuclear extract, it regulates the pattern, but not the efficiency, of splicing. In the absence of added ASF/SF2, splicing does not occur in HeLa cell S100 extract (cytoplasmic extract). Addition of ASF/SF2 to HeLa cell S100 extract activates splicing.

Cotranscriptional activation: Activation of RNA transcription by cofactors that modulate functional interactions between DNA-bound gene specific regulators and general transcription factors.

CTF: a transcriptional activator containing a proline-rich activation domain.

Deletion: the removal of a sequence of DNA, the regions on either side being joined together.

DNA: deoxyribonucleic acid. DNA is a long chain polymer which comprises the genetic material of most living organisms (some viruses have genes comprising ribonucleic acid, RNA). The repeating units in DNA polymers are four different nucleotides, each of which comprises one of the four bases, adenine, guanine, cytosine and thymine bound to a deoxyribose sugar to which a phosphate group is attached. Triplets of nucleotides, referred to as codons, in DNA molecules code for amino acid in a polypeptide. The term codon is also used for the corresponding (and complementary) sequences of three nucleotides in the mRNA into which the DNA sequence is transcribed.

GAL4AH: A fusion protein containing the DNA binding domain of GAL4 and a 15 amino acid peptide, amphipathic α-helix.

GST-VP16: This notation refers to both the plasmid, and the resulting recombinant protein translated from it. The recombinant protein contains the fully active bipartite activation domain encompassing VP 16 residues 413-490 fused to a GST molecule.

GST-A456: This notation refers to both the plasmid, and the resulting recombinant protein translated from it. The recombinant protein is a VP16 protein, containing a partially active domain which lacks the C-terminal 34 residues, fused to a GST molecule.

Δ456FP442: This notation refers to both the plasmid, and the resulting recombinant protein translated from it. The recombinant protein is a VP 16 protein, containing a C-terminal deletion of the activation domain (A456 noted above) in addition to a phenyalanine to proline point mutation at position 442 in the truncated derivative.

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

La antigen: a human autoantigen involved in RNA polymerase III transcription which also copurifies with PC4.

Mimetic: A molecule (such as an organic chemical compound) that mimics the activity of a protein, such as the activity of p52 and p75 which activates activator-dependent, but not basal, transcription by various activators. Peptidomimetic and organomimetic embodiments are within the scope of this term, whereby the three-dimensional arrangement of the chemical constituents of such peptido- and organomimetics mimic the three-dimensional arrangement of the peptide backbone and component amino acid sidechains in the peptide, resulting in such peptido- and organomimetics of the peptides having substantial specific activator activity. For computer modeling applications, a pharmacophore is an idealized, three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs”, in Klegerman & Groves, eds., 1993. Pharmaceutical Biotechnology, Interpharm Press: Buffalo Grove, Ill., pp. 165-174 and Principles of Pharmacology (ed. Munson. 1995), chapter 102 for a description of techniques used in computer assisted drug design. Example 31 describes other methods which can be used to generate mimetics.

p52 gene: A gene, the mutation of which is associated with abnormal mRNA transcription and/or pre-mRNA splicing, and may be seen in certain tumors, such as breast cancers, for example adenocarconimas of the breast. A mutation of the p52 gene may include nucleotide sequence changes, additions or deletions, including deletion of large portions or all of the p52 gene. The term “p52 gene” is understood to include the various sequence polymorphisms and allelic variations that exist within the population. This term relates primarily to an isolated coding sequence, but can also include some or all of the flanking regulatory elements and/or intron sequences.

p75 gene: A gene, the mutation of which is associated with abnormal mRNA transcription, and may be seen in certain tumors, such as breast cancers, for example breast adenocarcinomas of the breast. A mutation of the p75 gene may include nucleotide sequence changes, additions or deletions, including deletion of large portions or all of the p75 gene. The term “p75 gene” is understood to include the various sequence polymorphisms and allelic variations that exist within the population. This term relates primarily to an isolated coding sequence, but can also include some or all of the flanking regulatory elements and/or intron sequences.

p52 cDNA: A mammalian cDNA molecule which, when transfected into p52 cells, expresses the p52 protein. The p52 cDNA can be derived by reverse transcription from the mRNA encoded by the p52 gene and lacks internal non-coding segments and transcription regulatory sequences present in the p52 gene.

p75 cDNA: A mammalian cDNA molecule which, when transfected into p75 cells, expresses the p75 protein. The p75 cDNA can be derived by reverse transcription from the mRNA encoded by the p75 gene and lacks internal non-coding segments and transcription regulatory sequences present in the p75 gene.

p52 protein: The protein encoded by the p52 cDNA, the altered expression or mutation of which can predispose to altered mRNA transcription and/or altered pre-mRNA splicing, and the development of certain cancers, such as breast adenocarcinoma. This definition is understood to include the various sequence polymorphisms that exist, wherein amino acid substitutions in the protein sequence do not affect the essential functions of the protein.

p52 is the 52 kD protein present in USA-derived PC4-containing fractions that mediates activator-dependent, but not basal, transcription by various activators. p52 interacts directly with the VP16 activation domain and with components of the general transcription machinery. p52 significantly enhances the transcription by: acidic activation domains of GALA-AH and pseudorabies IE, the proline-rich activation domain of CTF, the glutamine-rich activation domain of Sp1 and the acidic activation domain of adenovirus E1A.

p75 protein: the protein encoded by the p75 cDNA, the altered expression or mutation of which can predispose to altered mRNA transcription, and the development of certain cancers, such as breast adenocarcinoma. This definition is understood to include the various sequence polymorphisms that exist, wherein amino acid substitutions in the protein sequence do not affect the essential functions of the protein.

p75 is the 75 kD protein present in USA-derived PC4-containing fractions, that mediates activator-dependent, but not basal, transcription by various activators including the acidic activation domain of GALA-AH. Less p75 coactivation is observed by the proline-rich activation domain of CTF and the acidic activation domain of pseudorabies IE. There is no significant enhancement of the transcription by the glutatmine rich activation domain of Sp1 or the acidic activation domain of adenovirus E1A in the presence of p75. p75 interacts directly with the VP16 activation domain and with components of the general transcription machinery.

Mutant p52 gene: a mutant form of the p52 gene, which in some (but not all) embodiments is associated with breast carcinoma.

Mutant p75 gene: a mutant form of the p75 gene which in some (but not all) embodiments is associated with breast carcinoma.

Mutant p52 RNA: the RNA transcribed from a mutant p52 gene.

Mutant p75 RNA: the RNA transcribed from a mutant p75 gene.

Mutant p52 protein: the protein encoded by a mutant p52 gene.

Mutant p75 protein: the protein encoded by a mutant p75 gene.

Oligonucleotide: A linear polynucleotide sequence of up to about 200 nucleotide bases in length, for example a polynucleotide (such as DNA or RNA) which is at least 6 nucleotides, for example at least 15, 50, 100 or even 200 nucleotides long.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

ORF: open reading frame. Contains a series of nucleotide triplets (codons) coding for amino acids without any termination codons. These sequences are usually translatable into protein.

PCR: polymerase chain reaction. Describes a technique in which cycles of denaturation, annealing with primer, and then extension with DNA polymerase are used to amplify the number of copies of a target DNA sequence.

Pharmaceutically acceptable carriers: The pharmaceutically acceptable carriers useful in this invention are conventional. Remington's Pharmaceutical Sciences, by E. W. Martin, Mack Publishing Co., Easton. PA. 15th Edition (1975), describes compositions and formulations suitable for pharmaceutical delivery of the nucleic acids and proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, ethanol, combinations thereof, or the like, as a vehicle. The carrier and composition can be sterile, and the formulation suits the mode of administration. For solid compositions (e.g., powder, pill, tablet, or capsule forms), conventional non-toxic solid carriers can include, for example, pharmaceutical grades of mannitol, lactose, starch, sodium saccharine, cellulose, magnesium carbonate, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The composition call be a liquid solution, suspension, emulsion, tablet, pill, capsule, sustained release formulation, or powder. The composition can be formulated as a suppository, with traditional binders and carriers such as triglycerides.

Probes and primers: Nucleic acid probes and primers may readily be prepared based on the amino acid sequences provided by this invention. A probe is an isolated nucleic acid attached to a detectable label or reporter molecule. Typical labels include radioactive isotopes, ligands, chemiluminescent agents, and enzymes. Methods for labeling and guidance in the choice of labels appropriate for various purposes are discussed, e.g., in Sambrook et al., in Molecular Cloning: A Laboratory Manual, Cold Spring (1989) and Ausubel et al. in Current Protocols in Molecular Biology. Greene Publishing Associates and Wiley-Intersciences (1987).

Primers are short nucleic acids, for example DNA oligonucieotides 15 nucleotides or more in length. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by the polymerase chain reaction (PCR) or other nucleic-acid amplification methods known in the art.

Methods for preparing and using probes and primers are described, for example, in Sambrook et al., 1989, Ausubel et al., 1987, and Innis et al., PCR Protocols, A Guide to Methods and Applications. 1990, Innis et al. (eds.), 21-27, Academic Press, Inc., San Diego, Calif. PCR primer pairs can be derived from a known sequence, for example, by using computer programs intended for that purpose such as Primer (Version 0.5, © 1991, Whitehead Institute for Biomedical Research, Cambridge, Mass.). One of skill in the art will appreciate that the specificity of a particular probe or primer increases with its length. Thus, for example, a primer comprising 20 consecutive nucleotides will anneal to a target with a higher specificity than a corresponding primer of only 15 nucleotides. Thus, in order to obtain greater specificity, probes and primers may be selected that comprise 20, 25, 30, 35, 40, 50 or more consecutive nucleotides.

PC4: positive cofactor 4. Primarily localized to the USA derived fraction from HeLa cell nuclear extracts. PC4 is a single- and double-stranded DNA binding protein that mediates activator-dependent transcription, in a TBP and TAF-dependent manner, but is not required for basal activity in an in vitro reconstituted transcription system. It acts as a general transcriptional coactivator for a variety of activators and, consistent with its role as an adapter, directly interacts both with activation domains of regulatory factors and with the general transcription factor TFIIA. All these PC4 activities are negatively regulated in vivo by phosphorylation.

pseudorabies IE: pseudorabies immediate early protein. A transcriptional activator which contains an acidic activation domain.

Purified: The term purified does not require absolute purity; rather, it is intended as a relative term. Thus, for example, a purified peptide preparation is one in which the peptide or protein is more enriched than the peptide or protein is in its natural environment within a cell. In one embodiment, a preparation is purified such that the protein or peptide represents at least 50% of the total peptide or protein content of the preparation.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Sample: Includes biological samples containing genomic DNA, RNA, or protein obtained from body cells, such as those present in peripheral blood, urine, saliva, tissue biopsy, surgical specimen, fine needle aspirates, amniocentesis samples, and autopsy material.

Sp1: a transcriptional activator containing a glutamine-rich activation domain.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Homologues or orthologs of the p52 and p75 proteins, and the corresponding cDNA sequences, will possess a relatively high degree of sequence identity when aligned using standard methods. This homology will be more significant when the orthologous proteins or cDNAs are derived from species which are more closely related (e.g., human and chimpanzee sequences), compared to species more distantly related (e.g., human and C. elegans sequences).

Typically, p52 and p75 orthologs are at least 50% identical at the nucleotide level and at least 50% identical at the amino acid level when comparing human p52 or p75 to an orthologous p52 or p75.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Nail. Acad. Sci. USA 85:2444, 1988; Higgins & Sharp, Gene, 73:23744, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al, J. Mol. Biol. 215:403-10, 1990, presents a detailed consideration of sequence aligment methods and homology calculations.

The NCBI Basic Local Alignment Search Tool (BLAST) (Altschul et al. J. Mol. Biol. 215:403-10, 1990) is available from several sources, including the National Center for Biotechnology Information (NCBI, Bethesda, Md.) and on the Internet, for use in connection with the sequence analysis programs blastp, blastn, blastx, tblastn and tblastx. It can be accessed at http://www.ncbi.nlm.nih.gov/BLAST/. A description of how to determine sequence identity using this program is available at http://www.ncbi.nlm.nih.gov/BLAST/blast_help.html.

Alternatively, one can align the sequences by hand, and then count the number of identical nucleic acids or amino acid residues between the sequences. The resulting value is divided by the total number of residues in the sequence of interest. Multiplying this number by 100 is the percent identity between the two sequences.

Homologues of the disclosed human p52 and p75 proteins typically possess at least 60% sequence identity counted over full-length alignment with the amino acid sequence of human p52 or p75 using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least 70%, at least 75%, at least 80%, at least 90%, at least 95%, at least 98%, or at least 99% sequence identity. When less than the entire sequence is being compared for sequence identity, homologues will typically possess at least 75% sequence identity over short windows of 10-20 amino acids, and may possess sequence identities of at least 85%, at least 90%, at least 95%, or at least 98% sequence identity, depending on their similarity to the reference sequence. Methods for determining sequence identity over such short windows are described at http://www.ncbi.nlm.nih.gov/BLAST/blast_FAQs.html.

One of ordinary skill in the art will appreciate that these sequence identity ranges are provided for guidance only; it is entirely possible that strongly significant homologues could be obtained that fall outside of the ranges provided. The present invention provides not only the peptide homologues that are described above, but also nucleic acid molecules that encode such homologues.

An alternative indication that two nucleic acid molecules are closely related is that the two molecules hybridize to each other under stringent conditions, as described in EXAMPLE 28. Specific binding agent: An agent that binds substantially only to a defined target. As used herein, the terms “p75 peptide specific binding agent” and “p52 peptide specific binding agent” includes anti-p75 or anti-p52 peptide antibodies and other agents that bind substantially only to the p75 and/or p52 peptides. The antibodies may be monoclonal or polyclonal antibodies that are specific for the p75 and/or p52 peptides, as well as immunologically effective portions (“fragments”) thereof. In one embodiment, the antibodies used in the present invention are monoclonal antibodies (or immunologically effective portions thereof) and may also be humanized monoclonal antibodies (or immunologically effective portions thereof). Immunologically effective portions of monoclonal antibodies include Fab, Fab′, F(ab′)₂, Fabc and Fv portions (for a review, see Better and Horowitz, Methods. Enzymol. 178:476-96, 1989). Anti-inhibitory peptide antibodies may also be produced using standard procedures described in a number of texts, including Antibodies, A Laboratory Manual by Harlow and Lane, Cold Spring Harbor Laboratory (1988).

The determination that a particular agent binds substantially only to the p75 and/or p52 peptides may readily be made by using or adapting routine procedures. One suitable in vitro assay makes use of the Western blotting procedure (described in many standard texts, including Antibodies, A Laboratory Manual by Harlow and Lane). Western blotting may be used to determine that a given p75 or p52 peptide binding agent, such as an anti-p52 or p75 peptide monoclonal antibody, binds substantially only to the p75 and/or p52 protein.

Therapeutically active molecule: A molecule which inhibits growth of tumor and cells, such as breast adenocarcinomas. Examples of protein based therapeutically active molecules are p52. p75, and fragments thereof. Therapeutically active molecules can also be made from nucleic acids. Examples of nucleic acid based therapeutically active molecules are antisense molecules, catalytic oligonucleotide sequences, triple strand nucleic acid molecules, gene therapy vectors containing the therapeutic p52 and/or p75 sequences, and circular nucleic acid molecules.

Transformed: A transformed cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Transgenic Cell: transformed cells which contain foreign, non-native DNA.

USA: upstream stimulatory activity (USA) fraction. This fraction is generated from a nuclear extract derived from human HeLa cells as described in Meisterernst and Roeder (Cell 67:557-67, 1991). The USA fraction is enriched at least four PCs: PC1, PC2, PC3, PC4.

VP16: a transcriptional activator containing an acidic activation domain.

V5 epitope: A 14 amino acid synthetic peptide, used to generate monoclonal antibodies. Purchased from Invitrogen (Carlsbad, Calif.).

Variant p75 peptides: Peptides having one or more amino acid substitutions, one or more amino acid deletions, and/or one or more amino acid insertions, so long as the peptide retains the property of a transcriptional co-activator. Conservative amino acid substitutions may be made in at least 1 position, for example 2, 3, 4, 5 or even 10 or more positions, as long as the peptide retains the activity of enhancing activated transcription, as readily measured by the in vitro transcription assay disclosed in the present specification (see EXAMPLE 5).

Variant p52 peptides: Peptides having one or more amino acid substitutions, one or more amino acid deletions, and/or one or more amino acid insertions, so long as the peptide retains the properties of a general co-activator of activated transcription and/or as a modulator of ASF/SF2 pre-mRNA splicing. Conservative amino acid substitutions may be made in at least 1 position, for example 2, 3, 4, 5 or even 10 or more positions, as long as the peptide retains the ability to function as a general co-activator that enhances activated transcription and the ability to modulate ASF/SF2 pre-mRNA splicing activity, as readily measured by the in vitro transcription assay (see EXAMPLE 5) and the in vitro splicing assay (see EXAMPLE 18) disclosed in the present specification.

Variants of Amino Acid and Nucleic Acid Sequences: The production of p52 or p75 proteins can be accomplished in a variety of ways (for example see EXAMPLES 4 and 20). DNA sequences which encode for the protein, or a fragment of the protein, can be engineered such that they allow the protein to be expressed in eukaryotic cells, bacteria, insects, and/or plants. In order to accomplish this expression, the DNA sequence can be altered and operably linked to other regulatory sequences. The final product, which contains the regulatory sequences and the therapeutic protein, is referred to as a vector. This vector can then be introduced into the eukaryotic cells, bacteria, insect, and/or plant. Once inside the cell the vector allows the protein to be produced.

One of ordinary skill in the art will appreciate that the DNA can be altered in numerous ways without affecting the biological activity of the encoded protein. For example, PCR may be used to produce variations in the DNA sequence which encodes p52 or p75. Such variants may be variants that are optimized for codon preference in a host cell that is to be used to express the protein, or other sequence changes that facilitate expression.

Two types of cDNA sequence variant may be produced. In the first type, the variation in the cDNA sequence is not manifested as a change in the amino acid sequence of the encoded polypeptide. These silent variations are simply a reflection of the degeneracy of the genetic code. In the second type, the cDNA sequence variation does result in a change in the amino acid sequence of the encoded protein. In such cases, the variant cDNA sequence produces a variant polypeptide sequence. In order to optimize preservation of the functional and immunologic identity of the encoded polypeptide, conservative amino acid substitutions may be made. Conservative substitutions replace one amino acid with another amino acid that is similar in size, hydrophobicity, etc. Such substitutions generally are conservative when it is desired to timely modulate the characteristics of the protein. Examples of amino acids which may be substituted for an original amino acid in a protein and which are regarded as conservative substitutions include: Ser for Ala; Lys for Arg; Gin or His for Asn; Glu for Asp; Ser for Cys; Asn for Gin; Asp for Glu; Pro for Gly; Asn or Gin for His; Leu or Val for Ile; Ile or Val for Leu; Arg or Gin for Lys; Leu or Ile for Met; Met, Leu or Tyr for Phe; Thr for Ser; Ser for Thr; Tyr for Trp; Trp or Phe for Tyr; and Ile or Leu for Val.

Variations in the cDNA sequence that result in amino acid changes, whether conservative or not, are minimized in order to preserve the optimal functional and immunologic identity of the encoded protein. The immunologic identity of the protein may be assessed by determining whether h is recognized by an antibody to p52 or p75; a variant that is recognized by such an antibody is immunologically conserved. In one embodiment, a cDNA sequence variant will introduce no more than 20, and for example fewer than 10 amino acid substitutions into the encoded polypeptide. Variant amino acid sequences can, for example, be 80%, 90% or even 95% identical to the native amino acid sequence.

Vector: A nucleic acid molecule as introduced into a host cell, thereby producing a transformed host cell. A vector may include nucleic acid sequences that permit it to replicate in the host cell, such as an origin of replication. A vector may also include one or more selectable marker genes and other genetic elements known in the art.

Tumor: A neoplasm.

Neoplasm: An abnormal growth of cells.

Cancer: A malignant neoplasm that has undergone characteristic anaplasia with loss of differentiation, increased rate of growth, invasion of surrounding tissue, and is capable of metastasis.

Malignant: cells which have the properties of anaplasia invasion and metastasis.

Normal cells: Non-tumor, non-malignant cells.

Mammal: This term includes both human and non-human mammals. Similarly, the term “subject” includes both human and veterinary subjects.

Additional definitions of common terms in molecular biology may be found in Lewin, B. “Genes V” published by Oxford University Press.

### EXAMPLE 1

### Cloning of p75 and p52

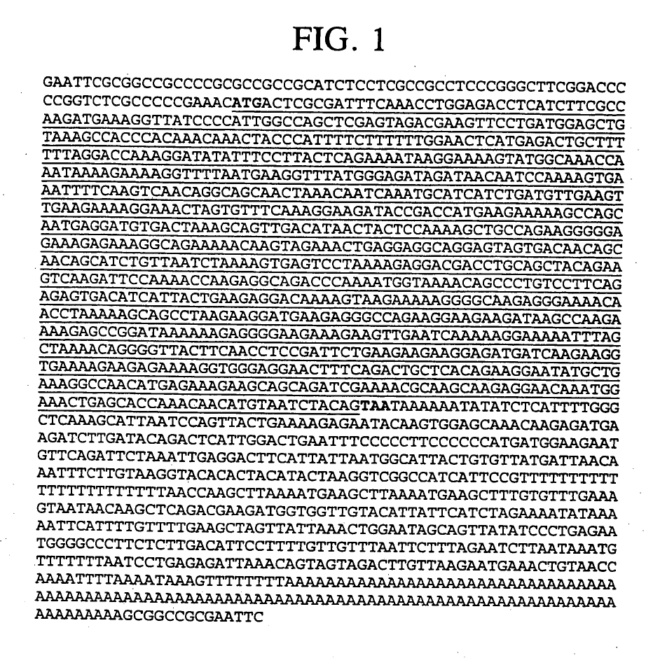

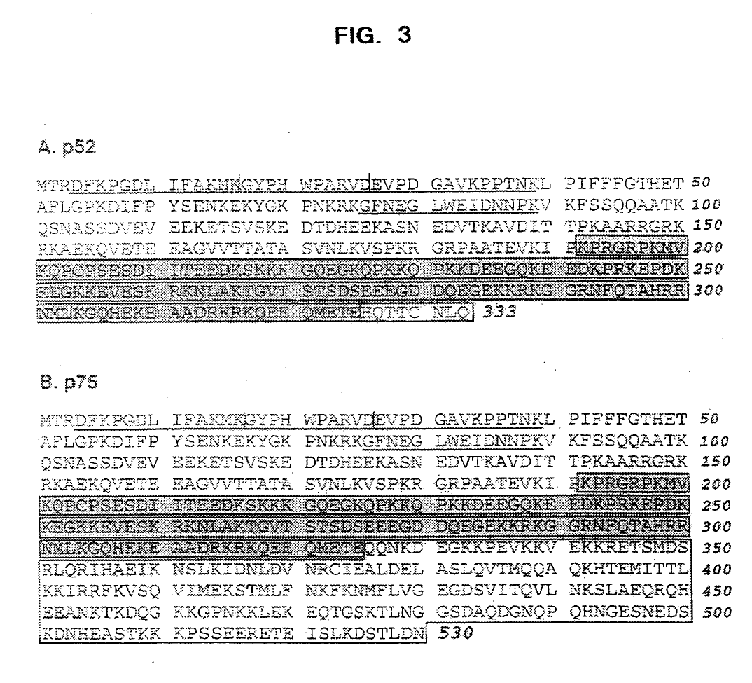

This example describes the cloning of p75 and p52 proteins. A protein of approximately 75 kD was observed to copurify with the general transcriptional coactivator PC4. To identify this protein, the 75 kDa polypeptide-containing Mono S fraction was resolved by SDS-PAGE and transferred to a nitrocellulose membrane. After visualization of the proteins by Ponceau S staining, the 75 kDa polypeptide was excised and subjected to N-terminal sequencing and in situ trypsin digestion for internal sequence analyses. One N-terminal, XXDFKPGDLIFAKMKGYPHXPAXVD (SEQ ID NO 11) and two internal, G/KYPT/HSPAS/RVDEVPDG/AAVKPPTNK (SEQ ID NO 12) and GFNEGLWEIDNNPK (SEQ ID NO 13) sequences were obtained. A degenerate oligonucleotide, 5′ GATTTCAARCCIGGIGATCTITTTGCIAARATGAARGGITACCCICA 3′ (SEQ ID NO 7), based on the N-terminal peptide sequence according to human codon bias, was used to screen a HeLa cDNA library in the lambda ZAPII vector. One of the resulting three positive clones contained a 1.8 kb insertion (SEQ ID NO 3) with a 333 amino acid open reading frame (SEQ ID NO 4) that represents an alternatively spliced isoform of p75 designated p52 (FIGS. 1 and 3A).

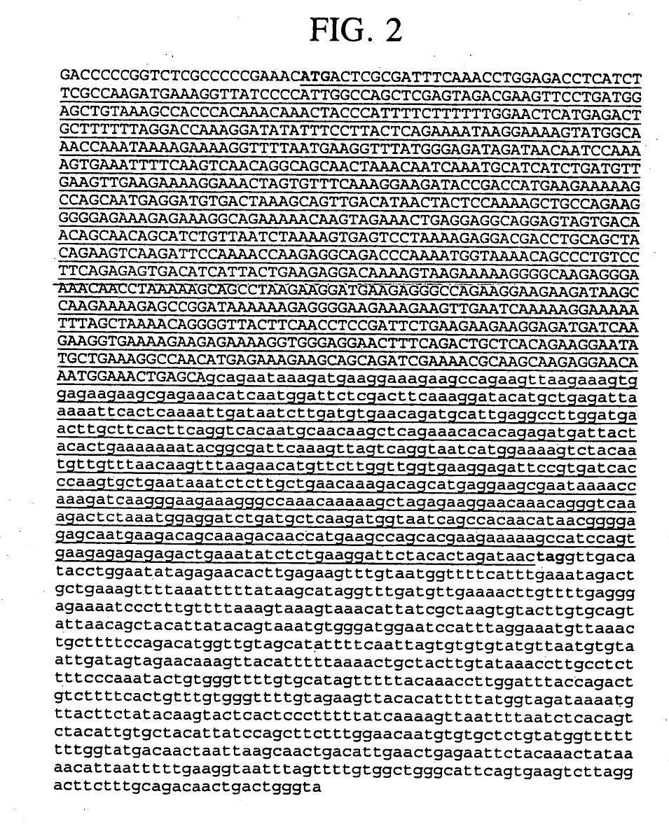

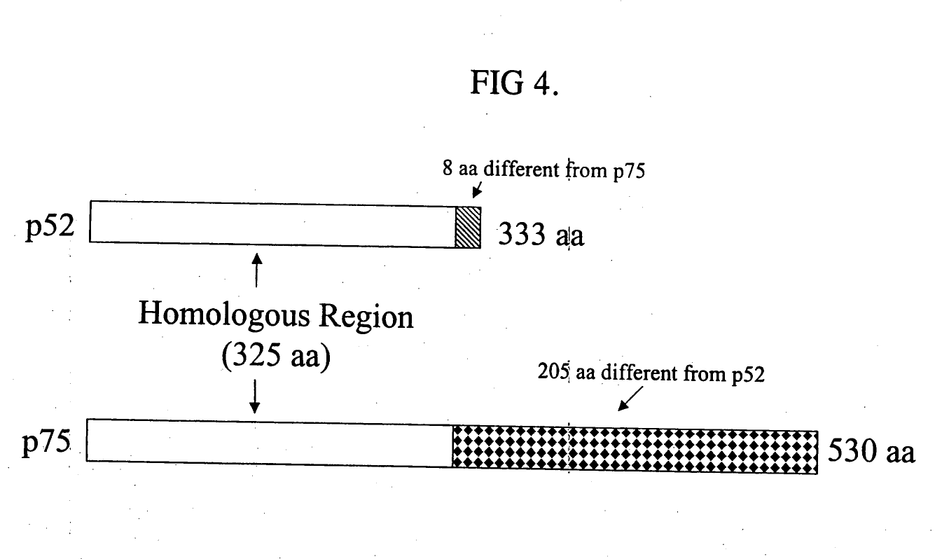

A second screen of the HeLa cDNA library with the 3′ coding region of p52 (Pst I-Bgl II fragment, from the coding sequence for amino acid residue 184 to 85 bp downstream of the stop codon) yielded 10 positive clones whose inserts have 3′ UTR sequences distinct from that of p52 cDNA. Although most had long 3′ UTR and poly A tails, none contained 5′ coding regions. The 5′ end of a 3.4 kb insertion corresponds to the sequence of p52 cDNA starting at bp 620, 5 bp upstream of unique Pst I site, but the sequence diverges from bp 1054, 22 bp upstream of the p52 stop codon. This different 3′ sequence generates an extended open reading frame of 530 amino acid residues (SEQ ID NOs 1 and 2, FIGS. 2 and 3B). The 5′ region of p75 was confirmed by PCR using a 5′ primer corresponding to 5′ UTR and start codon sequences of p52 cDNA and a 3′ primer corresponding to a unique sequence of p75 cDNA. FIG. 4 shows a comparison of the regions of homology between p52 and p75.

### EXAMPLE 2

### Northern Analysis of p75 and p52 RNA Expression

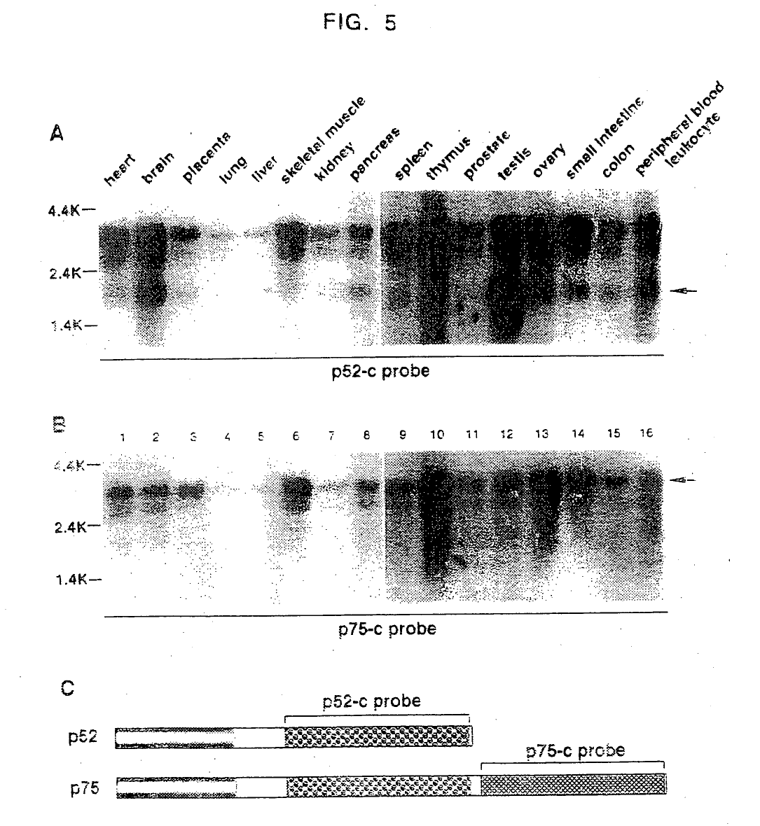

This example describes the Northern blot analyses of p52 and p75 RNA expression. Poly A+ RNAs isolated from human tissues (ClonTech, Palo Alto Calif.) were subjected to Northern analysis according to the manufacturer's instructions. The wash conditions were 0.2×SSC and 0.1% SDS for 20 minutes at 55° C. The 3′ half of the p52 coding region (Pst I-Bgl II fragment) (nucleotides 624-1.160 of SEQ ID NO 3) was used as the p52-C probe (FIG. 5C), while a 610 bp. PCR fragment corresponding to 3′ coding region of p75 (the last 610 nucleotides of SEQ ID NO 1) was used as the p75-C probe (FIG. 5C). Northern blot analyses revealed three major bands of 3.4 kb, 2.8 kb and 1.8 kb, with a probe corresponding to a C-terminal fragment of the p52 coding region (FIG. 5A), and two major bands of 3.4 kb and 2.8 kb with a probe corresponding to a C-terminal fragment of the p75 coding region (FIG. 5B). This indicates that the smallest species of mRNA of 1.8 kb corresponds to p52, whereas the other two larger species of mRNA of 3.4 kb and 2.8 kb correspond to p75. Both p52 and p75 mRNAs are ubiquitously expressed but the p52 mRNA is most abundant in the testis (FIG. 5A, lane 12), followed by thymus and brain (lanes 2 and 10), whereas the p75 mRNA is most abundant in thymus (FIG. 5B, lane 10). Expression of both p52 and p75 is minimal in the lung and liver.

### EXAMPLE 3

### Generation of Antibodies Against p52 and p75

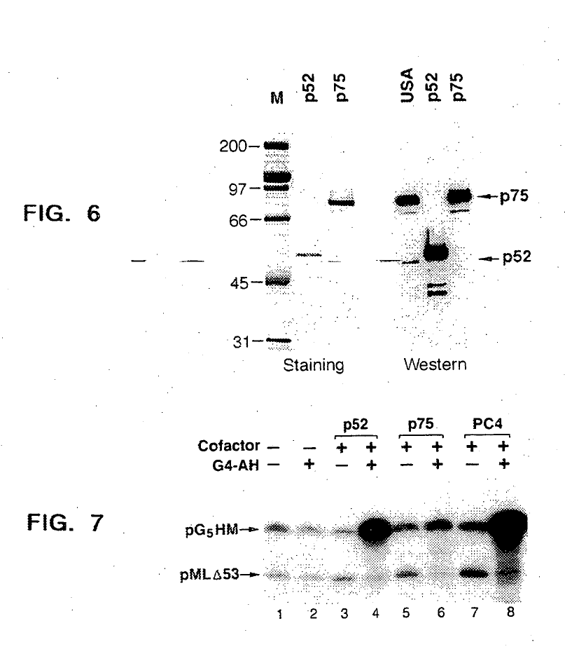

This example describes how polyclonal antibodies were generated which recognize both p52 and p75. Polyclonal antibodies were generated against the entire p52 sequence (333 amino acids) shown in SEQ ID NO 4. Purified recombinant p52 protein was used for the production of polyclonal antibodies by injecting NZW rabbits with continuous dorsal injections containing 0.1 μg of protein. This polyclonal antibody recognizes natural and recombinant p52 and p75 (FIG. 6).

To generate p52-specific antibodies, amino acids from the C-terminus of p52 (SEQ ID NO 6) can be used as the antigenic fragment, since this fragment is unique to p52. To generate p75-specific antibodies, amino acids from the C-terminus of p75 (SEQ ID NO 14 or fragments thereof) can be used as the antigenic fragment, since this fragment is unique to p75. The p52- and p75-specific antibodies can be generated using the above method, or alternatively using methods described in EXAMPLE 21.

### EXAMPLE 4

### Expression of Recombinant p52 and p75

This example describes the expression of recombinant p52 and p75 in E. coli. Both p52 and p75 cDNAs were introduced into a pET vector (Novagen, Milwaukee, Wis.) that introduced a six histidine tag (6H) and a heart muscle kinase (HMK) site at the N-terminus of each.

The Nru 1-EcoR V fragment of p52 cDNA was introduced into the Sma I site of the pGEX-2T(K) vector (Amersham Pharmacia Biotech, Piscataway, N.J.) to generate the plasmid GST-K-p52. The 6H(K)p52 plasmid was then generated by inserting an EcoR I fragment from GST-K-p52 into the EcoR I site of the pET11a-6H(K) vector, which includes sequences encoding six histidines and a HMK site. GST-K-p75 and 6H(K)p75 plasmids were created by replacing the Psi I-EcoR I fragment (627 bp) from either GST-K-p52 or 6H(K)p52 plasmid with the Pst 1-EcoR I fragment (1675 bp) from p75 cDNA.

The four plasmids described above (0.5 μg DNA) were transformed into BL21 E. coli cells and expressed by inducing with 1 mM IPTG for 3 hours as previously described (Ge and Roeder, Cell 78:513-23, 1994). After IPTG induction, bacteria were harvested and the 6H(K)p52 and 6H(K)p75 proteins were purified by subjecting the lysate to sequential chromatography. First the lysate was applied to a nickel NTA agarose affinity column, which has high affinity for the six histidine residues, and then eluted with 120 mM imidazole. This eluate was further purified by FPLC Mono S and Superdex 200 chromatography. The GST fusion proteins, GST-K-p52 and GST-K-p75, were purified by applying the lysate to a glutathione-Sepharose affinity column and eluting with 15 mM glutathione.

Polyclonal antibodies against recombinant p52 (see EXAMPLE 3) recognized the recombinant p52 and p75 proteins, as well as natural p52 and p75 proteins in the partially purified PC4-containing USA coactivator fraction, (FIG. 6, right panel). Thus the two cloned cDNAs encode the native p52 and p75 proteins.

### EXAMPLE 5

### In Vitro Transcription Assay

This example describes an in vitro transcription assay used to assess the coactivator functions of p52 and p75. This assay can be used to test the coactivator functions of p52 and/or p75 containing variant nucleic acid or amino acid sequences, p52 and/or p75 homologues and p52 and/or p75 mimetics. The standard in vitro transcription reaction uses reconstituted, highly purified general transcription factors. This system requires additional cofactors, either USA or derived components (Meisterernst et al., Cell, 66:981-93, 1991). In addition, PC4 alone can markedly enhance transcription by diverse activators (Ge and Roeder, Cell, 78:513-23, 1994; Kretzschmar et al., Cell, 78:525-34, 1994).

The in vitro transcription assay used is described in Ge et al. (Methods Enzymol. 274:57-71, 1996). Reconstituted, purified native, or recombinant general transcription factors were generated as described in Ge et al. (Methods Enzymol. 274:57-71, 1996). These transcription factors (0.1-0.5 pmole of each) were incubated at 30° C. for 1 hour in the presence or absence of the ³²P-labeled GAL4 DNA binding domain-activation domain fusion proteins (GST-fusion recombinant proteins, prepared as described in EXAMPLE 4 for GST-p52, also see Ge and Roeder, Cell 78:513-23, 1994) and with 50-500 ng of 6H(K)-tagged p52 or p75 (see EXAMPLE 4). The activated template (pG₅HMC2AT) contains five GALA DNA binding sites upstream of HIV-1 TATA box and adenovirus major late initiator elements linked to a 380 bp G-less cassette. The basal template (pMLΔ53) contains the adenovirus major late core promoter region (−53 to +10) linked to a 300 bp G-less cassette. These templates were radiolabeled by incubating them in: 20 mM HEPES, pH 8.2: 25 mg/ml BSA; 500 μM ATP/UTP; 25 μM CTP; and 5-10 pCi α-³²P-CTP. To determine the relative transcription activity, ³²P-labeled transcripts were subjected to denaturing polyacrylamide gel electrophoresis, visualized by autoradiography and quantitated using densitometry (Molecular Dynamics, Sunnyvale, Calif.).

As shown in FIG. 7, PC4 marginally stimulated basal level transcription in the absence of activator GALA-AH (lane 7), but markedly enhanced activated transcription on the pG₅HMC2AT reporter template (containing five GALA sites) in the presence of GALA-AH (lane 8). Like recombinant PC4, recombinant p52 and p75 had little or no effect on transcription in the absence of GAL4-AH (lanes 3 and 5). However, like PC4, p52 greatly enhanced transcription in the presence of GALA-AH (lane 4). p75 also enhanced transcription in the presence of GALA-AH (lane 6), but its effect was minimal (circa 3 fold) compared to p52 or PC4 (over 15 fold). These results indicate that recombinant p52 (and to a lesser extent p75) is a transcriptional coactivator capable of substituting for PC4 to potentiate GALA-AH-dependent transcription in vitro. “Enhanced transcription” in this example shall mean at least 2 fold increase, for example at least 3 fold.

### EXAMPLE 6

### p52 and p75 as General Coactivators

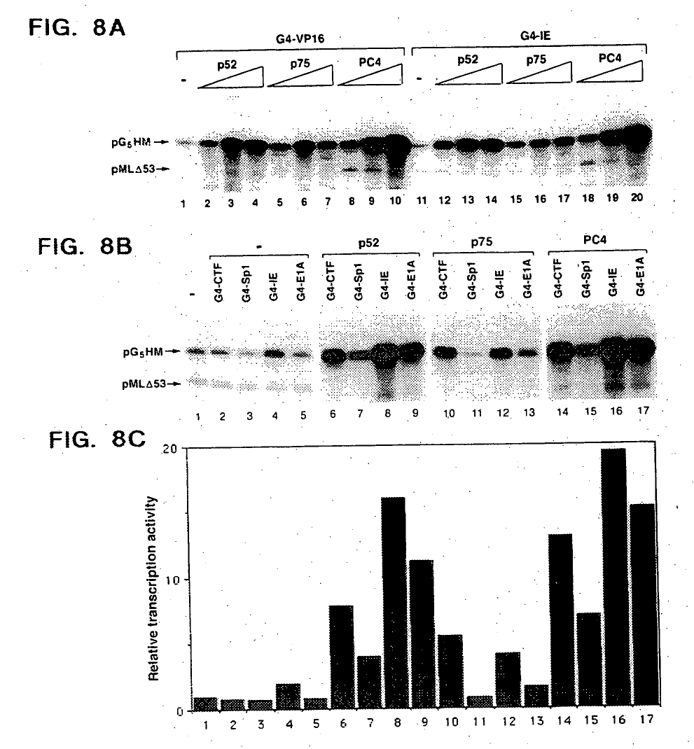

This example describes the use of the in vitro transcription assay described in EXAMPLE 5 to determine if p75 and/or p52 can act as general coactivators of transcription. This assay can be used to test the coactivator functions of p52 and/or p75 containing variant nucleic acid or amino acid sequences, p52 and/or p75 homologues and p52 and/or p75 mimetics. As shown in EXAMPLE 5, recombinant p52 and p75 (particularly p52) both can facilitate transcriptional activation by GAL4-AH. It was next determined whether, like PC4, they also could function as more general coactivators to potentiate activated transcription by other activators. Using the in vitro transcription assay described in EXAMPLE 5, p52 and p75 (4.59 pmoles or 13.5 pmoles) were incubated with 30 ng of transcriptional activator. Both p52 and p75 significantly enhanced activation both by the acidic activation domain of VP16 (FIG. 8A, lanes 2-7) and by the acidic activation domain (Martin et al., Genes Dev. 4:2371-82, 1990) of the pseudorabies immediate early protein (FIG. 8A, lanes 12-17) in a concentration-dependent manner (where the schematic ramp in FIG. 8A illustrates the increasing concentration of p52, p75 and PC4). FIG. 8B shows that recombinant p52 strongly stimulates transcriptional activation by GAL4 fusion proteins containing the proline-rich activation domain of CTF (lane 6 vs. lane 2), the glutamine-rich activation domain of Sp1 (lane 7 vs. lane 3), the activation domain of adenovirus EIA (lane 9 vs. lane 5) and, as shown above, the IE activation domain (lane 8 vs. lane 4). The quantitation of FIG. 8B is shown in FIG. 8C.

The coactivator functions observed with P52 closely parallel those of PC4 in the same assay (FIG. 8B, lanes 6-9 vs. lanes 14-17; see FIG. 8C for quantification). In contrast, p75 has only a moderate effect on activation by the proline-rich activation domain of CTF and the acidic activation domain of pseudorabies IE (FIG. 4B, lanes 10 and 12) and does not significantly enhance activation by either the glutamine-rich activation domain of Sp1 or the activation domain of EIA (lanes 11 and 13). Similar results were obtained when purified authentic Sp1 protein is tested in this system. Taken together, these observations demonstrate that recombinant p52 protein can act as a general transcriptional coactivator, comparable to PC4, whereas p75 functions less actively in potentiating activator function.

### EXAMPLE 7

### Protein-Protein Interactions

This example describes experiments conducted to determine if p52 and/or p75 bind to VP16 in vitro. This assay can be used to test the in vitro protein interactions of p52 and/or p75 containing variant nucleic acid or amino acid sequences, p52 and/or p75 homologues and p52 and/or p75 mimetics with VP16. The binding of recombinant p52 or p75 to immobilized GST-VP16 fusion proteins was assessed.

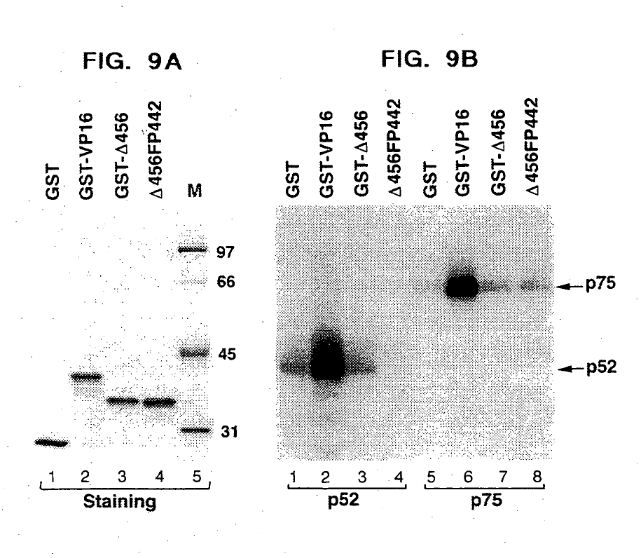

Recombinant ³²P-labeled 6H(K)p52 and 6H(K)p75 were prepared as described in EXAMPLE 4. Three different recombinant GST-VP16 constructs were generated (FIG. 9). The first contained the fully active bipartite activation domain encompassing VP16 residues 413-490 (GST-VP16). The second contained a partially active domain lacking the C-terminal 34 residues (GST-Δ456). The third contained an inactive domain containing an additional phenyalanine to proline point mutation at position 442 in the truncated derivative (Δ456FP442). These three plasmids were expressed in XA-90 E. coli cells, which were then induced with 1 mM IPTG for 3 hours to express the recombinant protein. The expressed fusion proteins were purified as described for the GST proteins in EXAMPLE 4.

Ten ng of ³² P-labeled (see EXAMPLE 9) 6H(K)p52 or 6H(k)p75, with 10-20 μg of each GST-VP16 fusion protein, was incubated at 4° C. for one hour in buffer A100 (20 mM HEPES-Na, pH 7.9: 10% glycerol: 0.2 mM EDTA; 100 mM KCl; 0.5 mM PMSF; 0.1% NP40 and 0.5 mg/ml BSA). The samples were then washed three times at 4° C. with buffer A200 (the same as A100 except that it contains 200 mM KCl) to reduce non-specific binding. Then 20% of the remaining bound proteins were analyzed by SDS-PAGE and detected by autoradiography. As shown in FIG. 9B, p52 and p75 both bound strongly to GST-VP16 (lanes 2 and 6). However, p52 and p75 bound only very weakly, and at levels close to the background levels observed with GST alone (lanes 1 and 5), to GST-A456 (lanes 3 and 7), and GST-A456FP442 (lanes 4 and 8). Thus the function of the VP16 activation domain in a p52/p75-dependent assay correlates well with its ability to bind p52/p75.

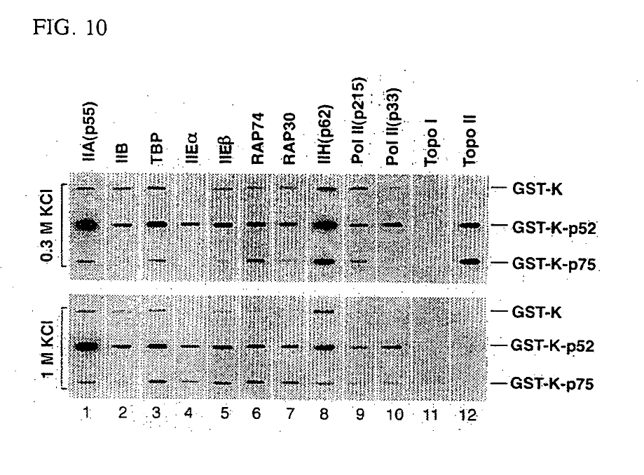

The interactions between p52 or p75 and components of the basal transcription machinery, were examined by testing the interactions of recombinant GST-K-p52 or GST-K-p75 with natural proteins in HeLa cell nuclear extract. GST-K-p52 or GST-K-p75 (50 μg of each, see EXAMPLE 4) immobilized on a glutathione Sepharose affinity column, were incubated with HeLa cell nuclear extract (500 ng, see EXAMPLE 15) for 14 hours at 4° C. The column was washed with BC100 (see EXAMPLE 13) to remove unbound, and non-specifically bound proteins. The remaining proteins were eluted with 2-3 volumes of 0.3 M KCl (which elutes 80-90% of the proteins), then 2-3 volumes of 1 M KCl. The eluted proteins were applied to a slot blot, which was then probed with antibodies against several transcription factors. ECL was used to visualize the proteins. Unexpectedly, all tested general transcription factors were bound to GST-K-p52 (but only few to GST-K-p75) column at levels significantly above the background levels observed for GST alone (FIG. 10).

### EXAMPLE 8

### Preparation of Other Recombinant Proteins

This example describes the preparation of other recombinant proteins. The p75-c protein was expressed in BL21 E. coli cells from the plasmid pET11d-p75-c in which a PCR fragment corresponding to the C-terminal coding region of p75 from amino acid residues ˜334 to 530 was inserted into pET11d vector. After 1 mM IPTG induction for 3 hours, the recombinant proteins were purified by applying the bacterial lysate to a Ni++agarose affinity column. Plasmids for expressing GST-ASF. GST-ARS and GST-RS were provided by J. Manley and S. Xiao, and the GST-fusion proteins were expressed and purified as described in EXAMPLE 4 (also see Ge and Roeder, Cell 78:513-23, 1994).

### EXAMPLE 9

### In Vitro Labeling of Proteins with HMK

This example describes the procedure for labeling proteins with ³²P using HMK. The labeling reaction is a 20 μl reaction containing: 0.5-1.0 μg of substrate protein; 20 mM Tris, pH 7.5; 100 mM NaCl; 1 mM DTT; 12 mM MgCl; 300 μCi γ-³²P-ATP; and 5 units HMK (Sigma, St. Louis, Mo.), which is incubated for 1 hour at 30° C. Upon the completion of the reaction, the labeled protein is passed over a G50 column to remove free nucleotides.

### EXAMPLE 10

### p52-PC4 In Vitro Interactions

This example describes an in vitro assay which assesses the ability of recombinant p52 to directly interact with PC4. This assay can be used to test the in vitro interactions of p52 and/or p75 containing variant nucleic acid or amino acid sequences, p52 and/or p75 homologues and p52 and/or p75 mimetics with PC4.

A 3-10 HeLa cell nuclear extract was prepared as described by Chiang et al. (EMBO J. 12:2749-62, 1993), and in EXAMPLE 15. This nuclear extract was passed over a phosphocellulose (P11) column, then eluted with 100 mM, 300 mM, 560 mM then 850 mM KCl generating individual fractions. These fractions were then subjected to Farwestern blot analyses (see EXAMPLE 13). After each fraction was resolved by SDS-PAGE and transferred to a PVDF membrane, renatured proteins were hybridized with GST-K-p52 or GST-K labeled by HMK (see EXAMPLE 9).

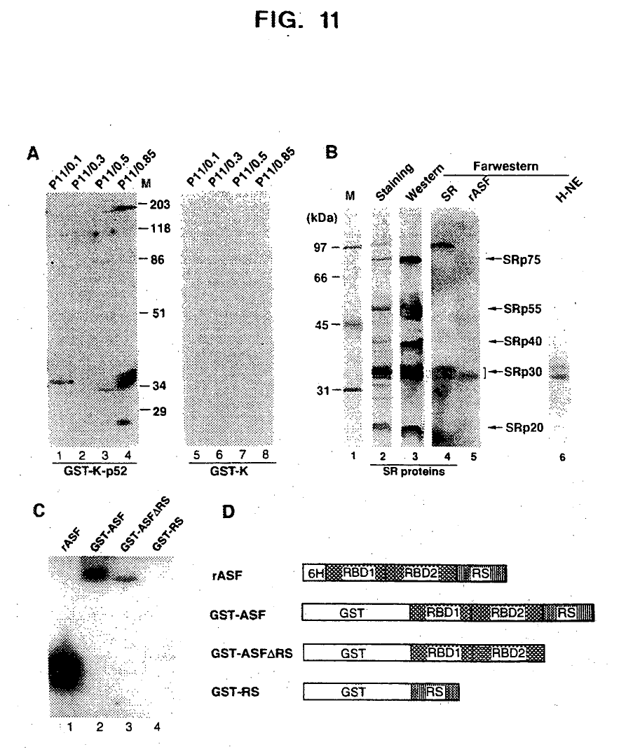

As shown in FIG. 11A, no interactions were detected when the membrane was blotted with the control probe GST-K (right panel). However, blotting with the GST-K-p52 probe (left panel) resulted in the detection of four specific polypeptides at: 20 kDa, 34 kDa (doublet) and 190 kDa, in the P11/0.85 M KCl fraction (lane 4). The P11/0.85 M KCl fraction contains the majority of the PC4 and TFIID activities. The 20 kDa protein was confirmed, by separate experiments, to be PC4. The identity of the 190 kDa protein is currently unknown. Of particular interest is the 34 kDa doublet, which migrated on the SDS-PAG like ASF/SF2, a splicing factor of the serine-arginine rich (SR) protein family (Ge et al., Cell 66:373-82, 1991; Krainer et al., Cell 66:383-94, 1991).

### EXAMPLE 11

### Identification of the 34 kD Doublet Protein

This example describes methods used to identify the 34 kD doublet observed in EXAMPLE 10. To confirm that the doublet was ASF/SF2, several different ASF/SF2-containing fractions, including purified SR proteins (from D. Derse and H. Chung, see Zahler et al., Genes Dev. 6:83747, 1992 for preparation), purified recombinant ASF/SF2 expressed in bacteria (6H-ASF/SF2 was prepared as described for p75-c in EXAMPLE 8) and HeLa cell nuclear extract (see EXAMPLE 15), were examined by Farwestern blot analysis (see EXAMPLE 13).

As shown in FIG. 11B, GST-K-p52 specifically interacted with a 34 kDa doublet corresponding to the SRp30 in the SR protein fraction purified from HeLa cells (lane 4) and recombinant ASF/SF2 (lane 5) as well as a 34 kDa doublet in the HeLa cell nuclear extract (lane 6). These observations demonstrate that p52 can indeed interact with ASF/SF2, but not other SR proteins, directly and specifically. However, in addition to ASF/SF2, p52 also interacts with a 100 kDa polypeptide (p100) copurified with SR proteins (lane 4). Since p100 can not be recognized by anti-SR antibody mAb104 (lane 3), it may not belong to the SR protein family.

### EXAMPLE 12

### Identification of ASF/SF2 Domains that Bind p52

This example describes the method used to determine which domain(s) of ASF/SF2 is required for p52 interaction. GST fused to the wild type ASF/SF2 (GST-ASF), the RNA-binding domains (GST-ARS) and the RS domain (GST-RS) of ASF/SF2 (see EXAMPLE 8 and FIG. 11D) were used for the Farwestern blot analysis (see EXAMPLE 13). After each fusion protein was resolved by SDS-PAGE and transferred, the renatured proteins were hybridized with GST-K-p52 labeled by HMK (see EXAMPLE 9).

As shown in FIG. 11C, p52 interacted strongly with wild type ASF/SF2 tagged with either six histidines (lane 1) or GST (lane 2) and weakly with GST-ARS (lane 3), but not at all with GST-RS (lane 4). This demonstrates ASF/SF2 uses distinct domains (RNA binding domains) to interact with the transcriptional coactivator p52, compared to the splicing factor U1 70K protein or other splicing factors, which use the RS domain (Wu and Maniatis, Cell 75:1061-70,1993; Eperon et al. EMBO J. 12:3607-17, 1993; Kohtz et al., Nature 368:119-24, 1994; Amrein et al., Cell 76:735-46, 1994).

A similar set of experiments can be conducted to identify the domains, or specific amino acids, of p52 essential for its interaction with ASF/SF2. Variant p52 peptides can be generated by constructing several p52 truncations as described above for ASF/SF2, or by random mutagenesis. These variant recombinant p52 proteins would then be subjected for Farwestern analysis with wild-type ASF/SF2. Those p52 mutants that show interactions with ASF/SF2 contain mutations in regions that are not essential for the ASF/SF2 interaction. In contrast, mutants that do not interact with ASF/SF2 contain mutations in regions that are probably important for the ASF/SF2 interaction. One region of p52 that is of particular interest are the highly charged C-terminal 134 amino acids (shaded residues in FIG. 3A). Greater than 50% of these amino acids are charged, indicating that they may play some role in protein-protein interactions.

### EXAMPLE 13

### Farwestern Blot Analysis

For Farwestern blot assays, protein samples were resolved by SDS-PAGE (12% polyacrylamide gel) and transferred to a PVDF membrane (Millipore). To denature the transferred proteins, the membrane was incubated in 6 M guanidine-HCl in buffer BC100 (20 mM Tris-Cl, pH 7.9; 10% glycerol, 0.1 M KCl; 0.2 mM EDTA, pH 8.0; 10 mM β-mercaptoethanol; and 0.5 mM PMSF) for 30 minutes. This was followed by renaturation of the proteins by successive treatment with 3.0, 1.5, 0.75, and 0.375 M guanidine-HCl in buffer BC100 for 10 minutes each at RT. The membrane was washed twice with buffer BC100, incubated in buffer BC100 containing 1% dry milk for one hour, followed by an incubation in buffer BC100 containing 1% dry milk and 10-20 ng/ml of ³²P-labeled GST-K or GST-K-p52 proteins, which were labeled by HMK (see EXAMPLE 9), for at least 10 hours at RT. The membrane was finally washed with 34 changes of buffer BC200 (BC100 buffer with 200 mM KCl) and the signals visualized by autoradiography.

### EXAMPLE 14

### p52-ASF/SF2 In Vivo Interactions

This example describes how co-immunoprecipitation assays were used to detect p52 interacting with ASF/SF2 in vivo. This-assay can also be used to test the in vitro interactions of p52 containing variant nucleic acid or amino acid sequences, p52 homologues and p52 mimetics with ASF/SF2. For co-immunoprecipitation assays, anti-p52 polyclonal antibodies (see EXAMPLE 3), were purified by GST-p52 affinity column and cross-linked to protein A sepharose beads. HeLa cell nuclear extract (4 ml, approximately 30 mg of protein) (see EXAMPLE 15) was adjusted to 0.5 M KCl and reloaded 4-5 times by gravity onto a 0.2 ml anti-p52 column. After extensively washing with buffer A500 (A100 buffer, see EXAMPLE 7, with 500 mM KCl), bound proteins were eluted with buffer A500 containing 100 mM glycine (pH 2.5) or A500 containing 100 mM triethylamine (pH 12) and precipitated with 10% TCA before immunoblot analysis using anti-p52 antibodies (see EXAMPLE 3) or a monoclonal antibody against SF2/ASF (from A. Krainer). Purified rabbit IgG (Amersham Pharmacia Biotech, Piscataway, N.J.) was cross-linked to protein A sepharose beads and used as a negative control.

For the transfection assay, an EcoR I-Pst I fragment released from p52 cDNA clone (pBS-p52) and a PCR fragment corresponding to p52 coding region from Pst I site to the stop codon were inserted into the pcDNA3.1/V5-HisB vector (containing a C-terminal lag encoding the V5 epitope and a polyhistidine metal-binding peptide) linearized by EcoR I and Xba I to generate a mammalian expression plasmid pcDNA3. 1-p52. Transient transfection of 293 cells was performed by using the standard calcium phosphate method. Nuclear extract (NE) from either untransfected or transfected 293 cells was prepared as described in EXAMPLE 15. Overexpressed protein and associated proteins were isolated using a Ni⁺⁺ agarose column. After incubation of NE with Ni⁺⁺ agarose resin, unbound materials were extensively washed with 0.3 M NaCl plus 20 mM imidazole, and the bound proteins were eluted with 0.5 M NaCl plus 1 M imidazole and detected by immunoblot analysis using a monoclonal antibody against V5 epitope or an anti-ASF/SF2 monoclonal antibody. Approximately 10-20% of overexpressed protein was recovered.

Association of p52 with ASF/SF2 in vivo was first demonstrated by co-immunoprecipitation assays. Proteins in the HeLa cell nuclear extract were precipitated either with anti-p52 antibody or with control IgG. The bound proteins were then monitored by immunoblot analyses. Because of their low abundance, in most cases p52, and p75, could not be directly detected in either HeLa or 293 cell nuclear extract. However, both p52 and p75 were greatly enriched in the partially purified PC4-containing fraction USA. Polyclonal antibodies against p52 specifically precipitated a protein with a relative molecular mass of 52 kDa together with a protein of 35 kDa, which was recognized by a monoclonal antibody against ASF/SF2 (A. Krainer). This result indicates that p52 is associated with ASF/SF2 in vivo. Since ASF/SF2 is much more abundant than p52, only ⁻1-2% of endogenous ASF/SF2 is associated with p52. It is important to note that the polyclonal antibody generated from recombinant p52 also recognized the p75 protein by immunoblot but could not precipitate p75 in the native condition. The properties of p52 and p75 appear to be distinct, even though p75 shares most of p52 coding sequence (FIG. 4).

Association of p52 with ASF/SF2 was also demonstrated using a transient transfection assay described above. The recombinant p52 protein was detected by using a monoclonal antibody against the V5 epitope. ASF/SF2 was detected by using the anti-ASF/SF2 monoclonal antibody. Transfected p52 was efficiently expressed (˜90% efficiency determined by immunofluorescence study) in 293 cells, a human embryonic kidney cell line. Both ASF/SF2 and overexpressed p52 from transfected cells bound to the nickel column but neither p52, nor ASF/SF2 from untransfected cells bound to the nickel column. Thus p52 interacts with ASF/SF2 in vivo.

### EXAMPLE 15

### Preparation of Cell Extracts

This example describes the generation of various extracts from HeLa and 293 cells, although the same methods can be used to generate cell extracts from other cell types (also see Ge and Manley, Cell 62:25-34, 1991 and Lee et al., Gene. Anal. Tech. 5:22-31, 1988). Cells were harvested and homogenized with a dounce homogenizer (Wheaton) on ice. This homogenized cell extract was centrifuged for 10 minutes at 2000 rpm to separate nuclei (pellet/nuclear fraction) from the cytoplasmic organelles (cytoplasmic fraction, or S100 fraction). The nuclear fraction was centrifuged at 15K rpm for 20 minutes, generating a pellet and supernatant. The pellet (nuclear fraction) contains mainly nuclei. This pellet was homogenized in 420 mM NaCl to break open the nuclei, releasing the nucleoli. This homogenized extract was centrifuged to remove insoluble materials. The resulting supernatant fraction (final nuclear extract, NE) was saved and dialyzed against 42 mM ammonium sulfate. This NE can be used directly, or from this NE, transcription factors can be further purified.

### EXAMPLE 16

### Immunofluorescence

This example describes the indirect immunofluorescence method used to identify the in vivo subcellular localization of endogenous p52 and ASF/SF2. Untransfected HeLa cells were grown on uncoated glass cover slips, washed with PBS (phosphate buffered saline) 3-4 times, fixed with 0.5% paraformadehyde in PBS for 20 minutes on ice, then followed by incubation with methanol for two minutes at room temperature. The fixed cells were rinsed three times with 3% BSA in PBS then incubated with primary antibody. The primary antibodies (diluted in PBS) were added to the cells at dilutions of: 1:100 of anti-ASF/SF2 (monoclonal antibody culture supernatant, A. Kramer), 1:2000 of anti-p52 (see EXAMPLE 3) and 1:200 of anti-La (from J. Steitz) then incubated for two hours at room temperature. The cells were washed with PBS three times and incubated with secondary antibodies for visualization of the primary antibody. Goat anti-rabbit IgG conjugated with FITC (Pierce) was used to visualize p52, anti-mouse IgG conjugated with rhodamine was used to visualize ASF/SF2, and anti-human IgG conjugated with rhodamine for La antibodies for one hour at room temperature. After extensively washing with PBS, mounted slides were observed on a Zeiss LSM410 confocal laser scanning microscope.

ASF/SF2 and p52 localized to speckle-like particles with a diffuse distribution throughout the nucleoplasm, consistent with the known distribution of splicing machinery (Zeng et al., EMBO J. 16:1401-12, 1997). Most particles were double-stained with the antibodies against ASF/SF2 and p52 resulting in a yellow color. On the other hand, La antigen, a factor involved in RNA polymerase III transcription which also copurifies with PC4 (Ge and Roeder, Cell 78:513-23, 1994), was localized in the nucleoplasm (see also Jimenez-Garcia and Spector, Cell 73:47-59, 1993), but did not co-localize with p52. These observations, in combination with the results from co-immunoprecipitation and transfection assays (see EXAMPLE 14), demonstrate that the majority of endogenous ASF/SF2 and p52 are associated with each other in the nucleus.

### EXAMPLE 17

### Sp1-Dependent In Vitro Transcription Assay

This example describes the Sp-1 dependent in vitro transcription assay. This assay can also be used to test the Sp-1 dependent in vitro transcription of p52 and/or p75 containing variant nucleic acid or amino acid sequences, p52 and/or p75 homologues and p52 and/or p75 mimetics. Reactions were reconstituted with partially purified general transcription factors (see Ge et al. Meth. Enzymol. 274:57-71, 1996) TFIIA, TFIIE/F/H and RNA polymerase II, affinity-purified TFIID (Flag-tagged), and recombinant TFIIB in the presence of template pHIV-WT (or called pMHIV-WT, Meisterernst et al., Cell 66:981-93, 1991), which contains HIV-1 promoter sequence from position −109 to −8 and ML initiator region from −7 to +9 linked with a 380 bp G-less cassette, for Sp1-activated transcription and pMLΔ53 for basal transcription as previously described (Ge et al., Meth. Enzymol. 274:57-71, 1996). These templates were ³²P-radiolabeled as described in EXAMPLE 6. A standard reaction (25 μl) was incubated at 30° C. for 60 minutes in the presence or absence of native Sp1 (5-20 ng) purified from HeLa cells (as in Jackson and Tjian, Proc. Natl. Acad. Sci. USA 86:1781-5, 1989) and the coactivators as indicated. ³²P-labeled transcripts were phenol/chloroform-extracted, ethanol-precipitated, analyzed by a 5% denaturing polyacrylamide gel and visualized by autoradiography. The relative transcription activity was determined by densitometry (Molecular Dynamics, Sunnyvale, Calif.).

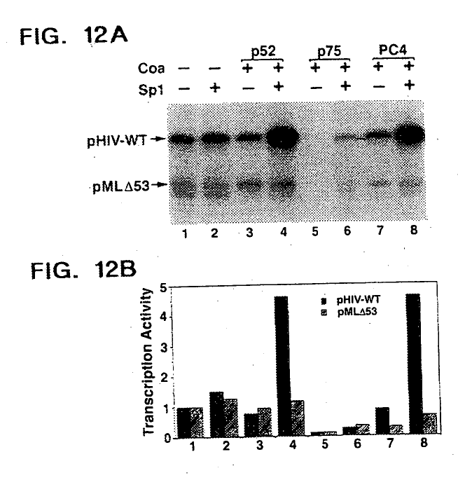

In an in vitro transcription system reconstituted with partially purified and recombinant general transcription factors, addition of both Sp1 (a natural activator, Kadonaga et al., Cell 51:1079-90, 1987) and recombinant 6H(K)p52 (see EXAMPLE 4) markedly enhanced Sp1-dependent transcription on the HIV-1 promoter-containing template (pHIV-WT), but not the basal level transcription on control template (pMLΔ53) (FIG. 12, lane 4). No significant effect was observed in the presence of Sp1 (lane 2) or p52 (lane 3) alone (see FIG. 12B for quantification). Similarly, addition of recombinant PC4 also significantly enhanced Sp1-activated transcription (lane 8) but not non-Sp1-dependent transcription (lane 7). In contrast, p75 did not enhance Sp1-activated transcription (lane 6). A similar effect was observed when GALA-Sp1 was used (FIG. 8B). These results indicate that p52 functions as a coactivator to potentiate activated transcription not only by GALA-fused activation domains but also by naturally purified cellular activators.

### EXAMPLE 18

### In Vitro Splicing Assay

This example describes two in vitro splicing assays. These assays can also be used to test the effect of p52 and/or p75 molecules containing variant nucleic acid or amino acid sequences, p52 and/or p75 homologues and p52 and/or p75 mimetics on splicing. Capped ³²P-labeled pre-mRNA substrate was prepared from linearized pSVi66 (see Ge et al. Cell 66:373-82, 1991). This pre-mRNA substrate was purified on a 5% polyacrylamide/8 M urea gel. The in vitro splicing reactions (25 μl) were carried out at 30° C. for 2 hours in a medium containing 5 mM HEPES-Na (pH 7.9), 0.6% polyvinyl alcohol, 400 μM ATP, 20 mM creatine phosphate, 2 mM MgCl₂. 2 mM DTT, 20 fmoles of ³²P-labeled pre-mRNA and 10 μl of HeLa cell nuclear extract, or 7.5 pi of HeLa cell S100 extract (see EXAMPLE 15), in the absence or presence of 1.5, 3.0 or 4.5 pmoles of recombinant p52, p75 or p75-c (amino terminus-truncated p75). (see EXAMPLES 4 and 8). Spliced products were extracted with RNAzol (Tel-Test, Inc), analyzed on a 5% polyacrylamide-8 M urea gel and visualized by autoradiography.

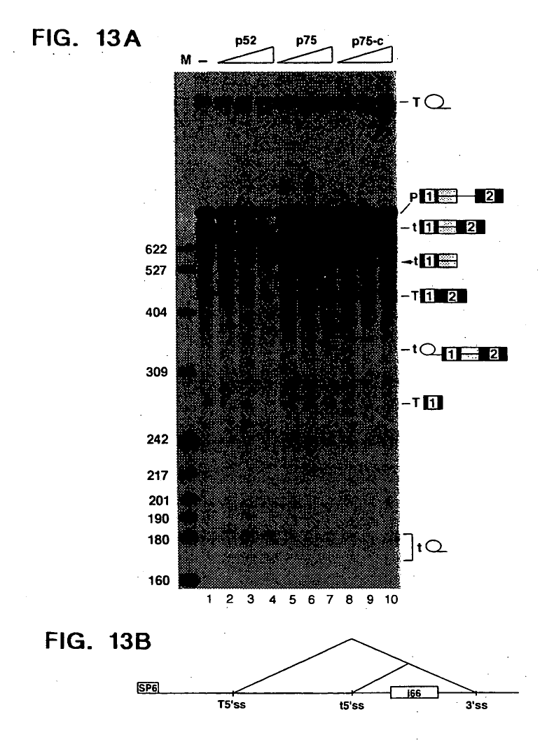

To determine the effect of p52 or p75 on splicing of SV40 early pre-mRNA transcribed from plasmid pSVi66 (Ge and Manley, Cell 62:25-34,1990; also see FIG. 13B), an in vitro splicing assay was used. Addition of recombinant p52 significantly enhanced the selection of the proximal small t 5′ splice site coupled with the reduced usage of the distal large T 5′ splice site (FIG. 13A, lanes 2-4). However, both p75 (lanes 5-7) and p75-c (lanes 8-10) had no influence on the splicing pattern or the splicing efficiency of same pre-mRNA. ASF/SF2 facilitates spliceosome assembly by promoting the binding of U 1 snRNP to the 5′ splice site (Eperon et al. EMBO J. 12:3607-17, 1993: Kohtz et al., Nature 368:119-24, 1994) and/or through direct binding to the 5′ splice site itself (Zuo and Manley, Proc. Natl. Acad. Sci. USA 91:3363-7, 1994). The observation that p52 preferentially enhances the first step of small t splicing is consistent with the fact that p52 affects pre-mRNA splicing by activating ASF/SF2, and subsequently mediates the early step of splicing. FIG. 13 clearly indicates that the p52-ASF/SF2 interaction can influence the 5′ splice site selection. Although p52 did not significantly affect splicing efficiency in this assay, this may be due to the limited amount of ASF/SF2 or other factors in the HeLa cell nuclear extract. Note that spliced t mRNA decreases, while t intron increases at a high concentration of p52 (FIG. 13A, lane 4). An explanation for this phenomenon is that recombinant p52 may be contaminated with trace amounts of RNase activity, which would favor degradation of linearized substrates, such as pre-mRNA and spliced mRNAs, rather than lariat introns.

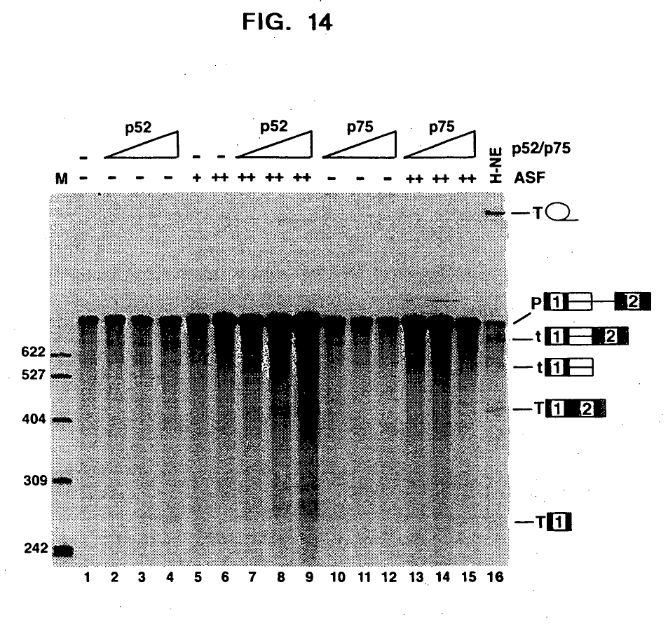

In addition to a role in alternative splicing, ASF/SF2 also functions as an essential splicing factor when added to an inactive HeLa cell S100 extract (Krainer et al., Genes Dev. 4:1158-71, 1990; Krainer et al., Cell 66:383-94, 1991; Ge et al., Cell 66:373-82, 1991). To test a direct functional relationship between p52 and ASF/SF2, both proteins were used in the S100 assay. In this assay, recombinant ASF/SF2 is added to HeLa cell S100 extract (see EXAMPLE 15) to activate splicing (FIG. 14). Addition of limited amounts of recombinant ASF/SF2 (lanes 5 and 6) or increasing amounts of recombinant p52 (lanes 24), or p75 (lanes 10-12), alone did not significantly activate splicing of SV40 early pre-mRNA in the presence of HeLa cell S100 extract. However, addition of increasing amounts (indicated by the upward ramps) of p52 (lanes 7-9), but not p75 (lanes 13-15), in the presence of limiting ASF/SF2 results in a proportional activation of splicing of SV40 early pre-mRNA. Taken together, these results indicate that p52-ASF/SF2 interaction is functionally important in vitro and in vivo, as it will facilitate the recruitment of ASF/SF2 to the active transcription site and increase the effective concentration of ASF/SF2 available to enhance splicing efficiency and/or splice site selection.

### EXAMPLE 19

### p52 and p75 Expression is Decreased in Breast Cancer Cells

Defects in proper pol II transcription has been implicated in carcinogenesis and the development of other diseases including xeroderma pigmentosum (for reviews see Kornberg, TIBS 21:325-6, 1996 and Reines et al., TIBS 21:351-5, 1996). To investigate the possibility that p52 and/or p75 may play a role in the development of cancer, p52 and p75 expression was investigated in several cell lines isolated from various carcinomas. The analysis described in this example can be used to analyze p52 and p75 expression levels in samples containing normal, neoplastic, tumorous, or cancerous (malignant) material.

The RNA and protein levels of p52 and p75 were determined in several cancer cell lines: MDA-MB-468, MCF7 and MDA-MB-231 (breast adenocarcinomas), HeLa. 293, and COS-7.

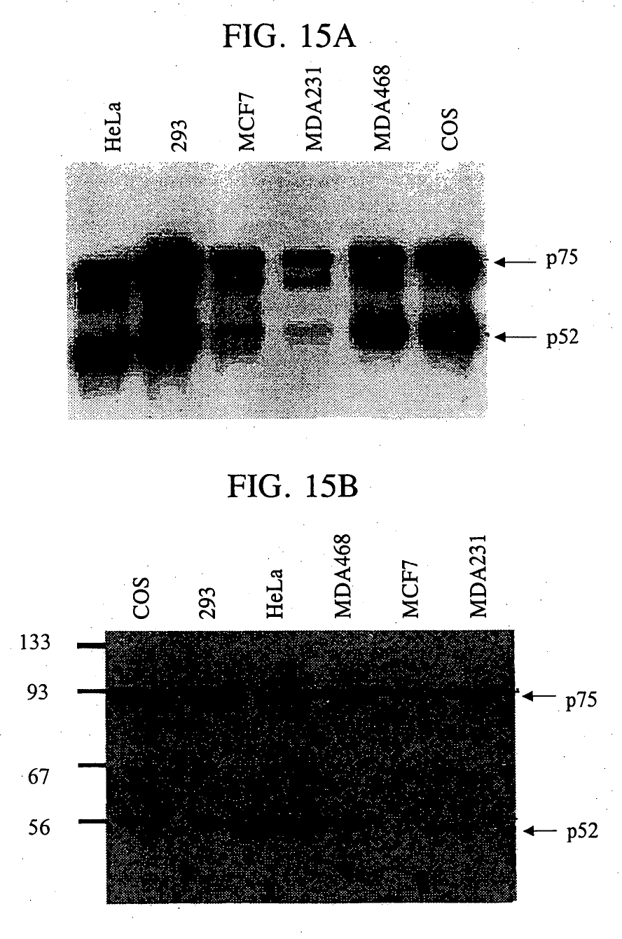

RNA was isolated from the cells, and Northern analysis was conducted as described in EXAMPLE 2, using the p52 probe shown in FIG. 5C. The total RNA analyzed in each lane was monitored by ethidium bromide staining of 28S and 18S ribosomal RNAs. As shown in FIG. 15A, the level of p52 RNA expression was dramatically decreased in all three breast cancer cell lines, relative to p52 RNA expression in other cancerous cell lines (HeLa, 293, and COS cells). The level of p75 RNA expression was also reduced in the breast cancer cells relative to the others, but to a lesser extent than p52.

Extracts containing cellular protein were prepared by lysing cells in SDS-PAGE loading buffer, such as: 50 mM TrisCl (pH 6.8). 100 mM dithiothreitol, 2% SDS, 0.1% bromophenol blue, and 10% glycerol. The proteins were subjected to SDS-PAGE and Western analysis using the anti-p52 antibodies described in EXAMPLE 3. As shown in FIG. 15B, the level of p52 protein in the three breast cancer cell lines is dramatically decreased relative to the level of p52 protein in cell lines from other origins. The amount of p75 protein expression was also reduced in the breast cancer cell line relative to the others, but to a lesser extent than p52. To control for total amount of protein loaded into each lane, the same blot was probed with an anti-TBP antibody, (TBP is an essential transcription factor).

Interestingly, the levels of both p52 and p75 RNA and protein expression correlate with the tumorigenicity. The cell line MDA-MD-231 is the most tumorigenic, and has the lowest levels of p52 and p75 RNA and protein. These results strongly suggest that both p52 and p75 play a role in tumorigenesis, such as tumorigenesis in breast cancers, and other cancers that can be determined by using the methods in this example.

### EXAMPLE 20

### Expression or p52 and p75 cDNA Sequences

With the provision of the human p52 and p75 cDNAs (SEQ ID NOs 3 and 1, respectively), the expression and purification of the corresponding p52 or p75 protein by standard laboratory techniques is now enabled. The purified protein may be used for functional analyses, antibody production, diagnosis, and patient therapy. Furthermore, the DNA sequence of the p52 and p75 cDNAs can be manipulated in studies to understand the expression of the gene and the function of its product. Mutant forms of p52 or p75 may be isolated based upon information contained herein, and may be studied in order to detect alteration in expression patterns in terms of relative quantities, tissue specificity and functional properties of the encoded mutant p52 and/or p75 proteins. Partial or full-length cDNA sequences, which encode for the subject protein, may be ligated into bacterial expression vectors. Methods for expressing large amounts of protein from a cloned gene introduced into E. coli may be utilized for the purification, localization and functional analysis of proteins. For example, fusion proteins consisting of amino terminal peptides encoded by a portion of the E. coli lacZ or trpE gene linked to p52 and p75 proteins may be used to prepare polyclonal and monoclonal antibodies against these proteins. Thereafter, these antibodies may be used to purify proteins by immunoaffinity chromatography, in diagnostic assays to quantitate the levels of protein and to localize proteins in tissues and individual cells by immunofluorescence.

Intact native protein may also be produced in E. coli in large amounts for functional studies. Methods and plasmid vectors for producing fusion proteins and intact native proteins in bacteria are described in Sambrook et al. (Molecular Cloning: A Laboratory Manual, Cold Spring Harbor, N.Y., 1989, chapter 17, herein incorporated by reference). Such fusion proteins may be made in large amounts, are easy to purify, and can be used to elicit antibody response. Native proteins can be produced in bacteria by placing a strong, regulated promoter and an efficient ribosome binding site upstream of the cloned gene. If low levels of protein are produced, additional steps may be taken to increase protein production; if high levels of protein are produced, purification is relatively easy. Suitable methods are presented in Sambrook et al. (Molecular Cloning: A Laboratory Manual, Cold Spring Harbor, N.Y., 1989) and are well known in the art. Often, proteins expressed at high levels are found in insoluble inclusion bodies. Methods for extracting proteins from these aggregates are described by Sambrook et al. (Molecular Cloning: A Laboratory Manual, Cold Spring Harbor, N.Y., 1989, Chapter 17).

Vector systems suitable for the expression of lacZ fusion genes include the pUR series of vectors (Ruther and Muller-Hill, 1983, EMBO J. 2:1791), pEXI-3 (Stanley and Luzio, 1984, EMBO J. 3:1429) and pMRI00 (Gray et al., 1982, Proc. Natl. Acad. Sci. USA 79:6598). Vectors suitable for the production of intact native proteins include pKC30 (Shimatake and Rosenberg, 1981, Nature 292:128), pKK177-3 (Amann and Brosius, 1985, Gene 40:183) and pET-3 (Studiar and Moffatt, 1986. J. Mol. Biol. 189:113). The p52 and/or p75 fusion proteins may be isolated from protein gels, lyophilized, ground into a powder and used as an antigen. The DNA sequence can also be transferred to other cloning vehicles, such as other plasmids, bacteriophages, cosmids, animal viruses and yeast artificial chromosomes (YACs) (Burke et al. 1987, Science 236:806-12). These vectors may then be introduced into a variety of hosts including somatic cells, and simple or complex organisms, such as bacteria, fungi (Timberlake and Marshall, 1989, Science 244:1313-7), invertebrates, plants (Gasser and Fraley, 1989, Science 244:1293), and mammals (Pursel et al., 1989, Science 244:1281-8), which cell or organisms are rendered transgenic by the introduction of the heterologous p52 or p75 cDNA.

For expression in mammalian cells, the cDNA sequence may be ligated to heterologous promoters, such as the simian virus SV40, promoter in the pSV2 vector (Mulligan and Berg, 1981, Proc. Natl. Acad. Sci. USA 78:2072-6), and introduced into cells, such as monkey COS-1 cells (Gluzman, 1981, Cell 23:175-82), to achieve transient or long-term expression. The stable integration of the chimeric gene construct may be maintained in mammalian cells by biochemical selection, such as neomycin (Southern and Berg, 1982, J. Mol. Appl. Genet. 1:327-41) and mycophoenolic acid (Mulligan and Berg, 1981, Proc. Natl. Acad. Sci. USA 78:2072-6).

DNA sequences can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR.

The cDNA sequence (or portions derived from it) or a mini gene (a cDNA with an intron and its own promoter) may be introduced into eukaryotic expression vectors by conventional techniques. These vectors are designed to permit the transcription of the cDNA eukaryotic cells by providing regulatory sequences that initiate and enhance the transcription of the cDNA and ensure its proper splicing and polyadenylation. Vectors containing the promoter and enhancer regions of the SV40 or long terminal repeat (LTR) of the Rous Sarcoma virus and polyadenylation and splicing signal from SV40 are readily available (Mulligan and Berg, 1981, Proc. Natl. Acad. Sci. USA 78:2072-6; Gorman et al., 1982, Proc. Natl. Acad. Sci USA 78:6777-81). The level of expression of the cDNA can be manipulated with this type of vector, either by using promoters that have different activities (for example, the baculovirus pAC373 can express cDNAs at high levels in S. frugiperda cells (Summers and Smith, 1985, Genetically Altered Viruses and the Environment, Fields et al. (Eds.) 22:319-328, Cold Spring Harbor Laboratory Press. Cold Spring Harbor, N.Y.) or by using vectors that contain promoters amenable to modulation, for example, the glucocorticoid-responsive promoter from the mouse mammary tumor virus (Lee et al., 1982, Nature 294:228). The expression of the cDNA can be monitored in the recipient cells 24 to 72 hours after introduction (transient expression).

In addition, some vectors contain selectable markers such as the gpt (Mulligan and Berg, 1981, Proc. Nail. Acad. Sci. USA 78:2072-6) or neo (Southern and Berg, 1982, J. Mol. Appl. Genet. 1:327-41) bacterial genes. These selectable markers permit selection of transfected cells that exhibit stable, long-term expression of the vectors (and therefore the cDNA). The vectors can be maintained in the cells as episomal, freely replicating entities by using regulatory elements of viruses such as papilloma (Sarver et al., 1981, Mol. Cell Biol. 1:486) or Epstein-Barr (Sugden et al., 1985, Mol. Cell Biol. 5:410). Alternatively, one can also produce cell lines that have integrated the vector into genomic DNA. Both of these types of cell lines produce the gene product on a continuous basis. One can also produce cell lines that have amplified the number of copies of the vector (and therefore of the cDNA as well) to create cell lines that can produce high levels of the gene product (Alt et al., 1978, J. Biol. Chem. 253:1357).

The transfer of DNA into eukaryotic, in particular human or other mammalian cells, is now a conventional technique. The vectors are introduced into the recipient cells as pure DNA (transfection) by, for example, precipitation with calcium phosphate (Graham and vander Eb. 1973, Virology 52:466) or strontium phosphate (Brash et al., 1987, Mol. Cell Biol. 7:2013), electroporation (Neumann et al., 1982, EMBO J. 1:841), lipofection (Felgner et al., 1987, Proc. Nail. Acad. Sci USA 84:7413), DEAE dextran (McCuthan et al., 1968, J. Natl. Cancer Inst. 41:351), microinjection (Mueller et al., 1978, Cell 15:579), protoplast fusion (Schafner, 1980, Proc. Nail. Acad. Sci. USA 77:2163-7), or pellet guns (Klein et al., 1987, Nature 327:70). Alternatively, the cDNA can be introduced by infection with virus vectors. Systems are developed that use, for example, retroviruses (Bernstein et al., 1985, Gen. Engrg. 7:235), adenoviruses (Ahmad et al., 1986, J. Virol. 57:267), or Herpes virus (Spaete et al., 1982, Cell 30:295).

These eukaryotic expression systems can be used for studies of the p52 and p75 genes and mutant forms of these genes, the p52 and p75 proteins and mutant forms of these proteins. Such uses include, for example, the identification of regulatory elements located in the 5′ region of the p52 and p75 genes on genomic clones that can be isolated from human genomic DNA libraries using the information contained in the present invention. The eukaryotic expression systems may also be used to study the function of the normal complete protein, specific portions of the protein, or of naturally occurring or artificially produced mutant proteins. Naturally occurring mutant proteins may exist in a variety of cancers or diseases, while artificially produced mutant proteins can be designed by site directed mutagenesis as described above. These latter studies may probe the function of any desired amino acid residue in the protein by mutating the nucleotide coding for that amino acid.

Using the above techniques, the expression vectors containing the p52 or p75 gene or cDNA sequence or fragments or variants or mutants thereof can be introduced into human cells, mammalian cells from other species or non-mammalian cells as desired. The choice of cell is determined by the purpose of the treatment. For example, monkey COS cells (Gluzman, 1981, Cell 23:175-82) that produce high levels of the SV40 T antigen and permit the replication of vectors containing the SV40 origin of replication may be used. Similarly, Chinese hamster ovary (CHO), mouse NIH 3T3 fibroblasts or human fibroblasts or lymphoblasts may be used.

One method that can be used to express the p52 or p75 polypeptide from the cloned p52 or p75 cDNA sequences in mammalian cells is to use the cloning vector, pXT1. This vector is commercially available from Stratagene (La Jolla, Calif.), contains the Long Terminal Repeats (LTRs) and a portion of the GAG gene from Moloney Murine Leukemia Virus. The position of the viral LTRs allows highly efficient, stable transfection of the region within the LTRs. The vector also contains the Herpes Simplex Thymidine Kinase promoter (TK), active in embryonal cells and in a wide variety of tissues in mice, and a selectable neomycin gene conferring G418 resistance. Two unique restriction sites BglII and XhoI are directly downstream from the TK promoter. p52 or p75 cDNA, including the entire open reading frame for the p52 or p75 protein and the 3′ untranslated region of the cDNA is cloned into one of the two unique restriction sites downstream from the promoter.

The ligated product is transfected into mouse NIH 3T3 cells using Lipofectin (Life Technologies, Inc., Rockville, Md.) under conditions outlined in the product specification. Positive transfectants are selected after growing the transfected cells in 600 μg/ml G418 (Sigma, St. Louis, Mo.). The protein is released into the supernatant and may be purified by standard immunoaffinity chromatography techniques using antibodies raised against the p52 or p75 protein (see EXAMPLES 3 and 21).

Expression of the p52 and/or p75 protein in eukaryotic cells can be used as a source of proteins to raise antibodies. The p52 and p75 proteins may be extracted following release of the protein into the supernatant as described above, or, the cDNA sequence may be incorporated into a eukaryotic expression vector and expressed as a chimeric protein with, for example, β-globin. Antibody to β-globin is thereafter used to purify the chimeric protein. Corresponding protease cleavage sites engineered between the β-globin gene and the cDNA are then used to separate the two polypeptide fragments from one another after translation. One useful expression vector for generating β-globin chimeric proteins is pSG5 (Stratagene, La Jolla, Calif.). This vector encodes rabbit β-globin.

The present invention thus encompasses recombinant vectors which comprise all or part of the p52 or p75 gene or cDNA sequences, for expression in a suitable host. The p52 or p75 DNA is operatively linked in the vector to an expression control sequence in the recombinant DNA molecule so that the p52 or p75 polypeptide can be expressed. The expression control sequence may be selected from the group consisting of sequences that control the expression of genes of prokaryotic or eukaryotic cells and their viruses and combinations thereof. The expression control sequence may be specifically selected from the group consisting of the lac system, the trp system, the tac system, the trc system, major operator and promoter regions of phage lambda, the control region of fd coat protein, the early and late promoters of SV40, promoters derived from polyoma, adenovirus, retrovirus, baculovirus and simian virus, the promoter for 3-phosphoglycerate kinase, the promoters of yeast acid phosphatase, the promoter of the yeast alpha-mating factors and combinations thereof.

The host cell, which may be transfected with the vector of this invention, may be selected from the group consisting of: E. coli, Pseudomonas, Bacillus subtilis, Bacillus stearothermophilus or other bacilli; other bacteria; yeast; fungi; plant; insect: mouse or other animal; or human tissue cells.

It is appreciated that for mutant or variant p52 or p75 DNA sequences, similar systems are employed to express and produce the mutant or variant product.

### EXAMPLE 21

### Production of p52 and p75 Antibodies

Monoclonal or polyclonal antibodies may be produced to either the normal p52 or p75 protein or mutant forms of these proteins. Antibodies raised against the full-length p52 peptide (SEQ ID NO 4) are likely to recognize both p52 and p75, because of the large number of identical amino acids between them. Antibodies which specifically recognize only p52 can be generated by using the C-terminal amino acid residues (SEQ ID NO 6) as an antigen, since these residues are unique to p52. Antibodies which specifically recognize only p75 can be generated by using the C-terminal amino acid residues (SEQ ID NO 14) as an antigen, which are unique to p75. Fragments of SEQ ID NO14 can also be used to generate p75-specific antibodies.

Optimally, antibodies raised against the p52 protein would specifically detect the p52 protein while antibodies raised against the p75 protein would specifically detect the p75 protein. That is, such antibodies would recognize and bind the protein and would not substantially recognize or bind to other proteins found in human cells. The determination that an antibody specifically detects the p52 or p75 protein is made by any one of a number of standard immunoassay methods; for instance, the Western blotting technique (Sambrook et al., 1989, Molecular Cloning: A Laboratory Manual. Cold Spring Harbor Laboratory, Cold Spring Harbor, N.Y.). To determine that a given antibody preparation (such as one produced in a mouse) specifically detects the p52 or p75 protein by Western blotting, total cellular protein is extracted from human cells (for example, lymphocytes) and electrophoresed on a sodium dodecyl sulfate-polyacrylamide gel. The proteins are then transferred to a membrane (for example, nitrocellulose) by Western blotting, and the antibody preparation is incubated with the membrane. After washing the membrane to remove non-specifically bound antibodies, the presence of specifically bound antibodies is detected by the use of an anti-mouse antibody conjugated to an enzyme such as alkaline phosphatase; application of the substrate 5-bromo-4-chloro-3-indolyl phosphate/nitro blue tetrazolium results in the production of a dense blue compound by immuno-localized alkaline phosphatase. Antibodies which specifically detect the p52 or p75 protein will, by this technique, be shown to bind to the p52 or p75 protein band (which will be localized at a given position on the gel determined by its molecular weight). Non-specific binding of the antibody to other proteins (such as serum albumen) may occur and may be detectable as a weak signal on the Western blot. The non-specific nature of this binding will be recognized by one skilled in the art by the weak signal obtained on the Western blot relative to the strong primary signal arising from the specific antibody-p52 or -p75 protein binding.

Substantially pure p52 or p75 protein suitable for use as an immunogen is isolated as already described. Concentration of protein in the final preparation is adjusted, for example, by concentration on an Amicon filter device, to the level of a few micrograms per milliliter. Monoclonal or polyclonal antibody to the protein can then be prepared.

Monoclonal Antibody Production by Hybridoma Fusion

Monoclonal antibody to epitopes of the p52 (for example SEQ ID NOs 4 or 6) or p75 (SEQ ID NOs 2 or 14) protein identified and isolated as described can be prepared from murine hybridomas according to the classical method of Kohler and Milstein (Nature 256:495, 1975) or derivative methods thereof. Briefly, a mouse is repetitively inoculated with a few micrograms of the selected protein over a period of a few weeks. The mouse is then sacrificed, and the antibody-producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall (Enzymol. 70:419, 1980), and derivative methods thereof. Selected positive clones can be expanded and their monoclonal antibody product harvested for use. Detailed procedures for monoclonal antibody production are described in Harlow and Lane (Antibodies: A Laboratory Manual. 1988, Cold Spring Harbor Laboratory, New York).

Polyclonal Antibody Production by Immunization

Polyclonal antiserum containing antibodies to heterogeneous epitopes of a single protein can be prepared by immunizing suitable animals with the expressed protein (for example see EXAMPLES 4 and 20), which can be unmodified or modified to enhance immunogenicity. Effective polyclonal antibody production is affected by many factors related both to the antigen and the host species. For example, small molecules tend to be less immunogenic than others and may require the use of carriers and adjuvant. Also, host animals vary in response to site of inoculations and dose, with both inadequate or excessive doses of antigen resulting in low titer antisera. Small doses (ng level) of antigen administered at multiple intradermal sites appears to be most reliable. An effective immunization protocol for rabbits can be found in Vaitukaitis et al. (J. Clin. Endocrinol. Metab. 33:988-91, 1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony et al. (In: Handbook of Experimental Immunology, Wier, D. (ed.). Chapter 19. Blackwell. 1973). Plateau concentration of antibody is usually in the range of 0.1 to 0.2 mg/ml of serum (about 12 μM). Affinity of the antisera for the antigen is determined by preparing competitive binding curves, as described, for example, by Fisher (Manual of Clinical Immunology, Chapter 42, 1980).

Labeled Antibodies

Antibodies of the present invention can be conjugated with various labels for their direct detection (see Chapter 9, Harlow and Lane, Antibodies: A Laboratory Manual. 1988). The label, which may include, but is not limited to, a radiolabel enzyme, fluorescent probe, or biotin, is chosen based on the method of detection available to the user.

Antibodies can be radiolabeled with iodine (¹²⁵I), which yields low-energy gamma and X-ray radiation. Briefly, 10 μg of protein in 25 μl of 0.5 M sodium phosphate (pH 7.50 is placed in a 1.5 ml conical tube. To this, 500 μC of Na¹²⁵I, and 25 μl of 2 mg/ml chloramine T is added and incubated for 60 sec at room temperature. To stop the reaction, 50 μl of chloramine T stop buffer is added (2.4 mg/ml sodium metabisulfite, 10 mg/ml tyrosine, 10% glycerol, 0.1% xylene cyanol in PBS). The iodinated antibody is separated from the iodotyrosine on a gel filtration column. Antibodies of the present invention can also be labeled with biotin, with enzymes such as alkaline phosphatase (AP) or horseradish peroxidase (HRP) or with fluorescent dyes. The method of producing these conjugates is determined by the reactive group on the label added.

### EXAMPLE 22

### Diagnostic Methods

An embodiment of the present invention is a method for screening a subject to determine if the subject carries a mutant p52 or p75 gene, or has heterozygous or homozygous deletions of the p52 or p75 gene, or if the gene has been partially or completely deleted. One major application of the p52 and p75 sequence information presented herein is in the area of genetic testing for predisposition to breast cancer owing to p52 and/or p75 deletion or mutation. The gene sequence of the p52 and p75 genes, including intron-exon boundaries is also useful in such diagnostic methods. The method comprises the steps of: providing a biological sample obtained from the subject, in which sample includes DNA or RNA, and providing an assay for detecting in the biological sample the presence of a mutant p52 or p75 gene, a mutant p52 or p75 RNA, a homozygously or heterozygously deleted p52 or p75 gene, or the absence, through deletion, of the p52 or p75 gene and corresponding RNA. Suitable biological samples include samples obtained from body cells, such as those present in peripheral blood, urine, saliva, tissue biopsy, surgical specimen, fine needle aspirate specimen, amniocentesis samples and autopsy material. The detection in the biological sample may be performed by a number of methodologies, as outlined below.

The foregoing assay may be assembled in the form of a diagnostic kit and can comprise either: hybridization with oligonucleotides: PCR amplification of the gene or a part thereof using oligonucleotide primers; RT-PCR amplification of the RNA or a part thereof using oligonucleotide primers; or direct sequencing of the p52 or p75 gene of the subject's genome using oligonucleotide primers. The efficiency of these molecular genetic methods should permit a rapid classification of patients affected by deletions or mutations of the p52 or p75 gene.

One embodiment of such detection techniques is the polymerase chain reaction amplification of reverse transcribed RNA (RT-PCR) of RNA isolated from cells (for example lymphocytes) followed by direct DNA sequence determination of the products. The presence of one or more nucleotide differences between the obtained sequence and the cDNA sequences, and especially, differences in the ORF portion of the nucleotide sequence are taken as indicative of a potential p52 or p75 gene mutation.

Alternatively, DNA extracted from lymphocytes or other cells may be used directly for amplification. The direct amplification from genomic DNA would be appropriate for analysis of the entire p52 or p75 gene including regulatory sequences located upstream and downstream from the open reading frame. Recent reviews of direct DNA diagnosis have been presented by Caskey (Science 236:1223-1228, 1989) and by Landegren et al. (Science 242:229-37, 1989).

Further studies of p52 or p75 genes isolated from subjects may reveal particular mutations, or deletions, which occur at a high frequency within this population of individuals. In this case, rather than sequencing the entire p52 or p75 gene, it may be possible to design DNA diagnostic methods to specifically detect the most common p52 or p75 mutations or deletions.

The detection of specific DNA mutations may be achieved by methods such as hybridization using specific oligonucleotides (Wallace et al., 1986, Cold Spring Harbor Symp. Quant. Biol. 51:257-61), direct DNA sequencing (Church and Gilbert, 1984, Proc. Natl. Acad. Sci. USA. 81:1991-5), the use of restriction enzymes (Flavell et al., 1978, Cell 15:25; Geever et al., 1981, Proc. Nail. Acad. Sci USA 78:5081), discrimination on the basis of electrophoretic mobility in gels with denaturing reagent (Myers and Maniatis, 1986, Cold Spring Harbor Symp. Quant. Biol. 51:275-284), RNase protection (Myers et al., 1985, Science 230:1242), chemical cleavage (Cotton et al., 1985, Proc. Nail. Acad. Sci. USA 85:4397-401), and the ligase-mediated detection procedure (Landegren et al., 1988, Science 241:1077).

Oligonucleotides specific to normal or mutant sequences are chemically synthesized using commercially available machines, labeled radioactively with isotopes (such as ³²P) or non-radioactively, with tags such as biolin (Ward and Langer et al., 1981. Proc. Nail. Acad. Sci. USA 78:6633-57), and hybridized to individual DNA samples immobilized on membranes or other solid supports by dot-blot or transfer from gels after electrophoresis. The presence of these specific sequences are visualized by methods such as autoradiography or fluorometric (Landegren et al., 1989, Science 242:229-37) or calorimetric reactions (Gebeyehu et al., 1987, Nucleic Acids Res. 15:4513-34). The absence of hybridization would indicate a mutation in the particular region of the gene, or a deleted p52 or p75 gene.

Sequence differences between normal and mutant forms of the p52 or p75 gene may also be revealed by the direct DNA sequencing method of Church and Gilbert (Proc. Natl. Acad. Sci. USA 81:1991-5, 1988). Cloned DNA segments may be used as probes to detect specific DNA segments. The sensitivity of this method is greatly enhanced when combined with PCR (Wrichnik et al., 1987, Nucleic Acids Res. 15:529-42; Wong et al., 1987, Nature 330:384-6; Stofle (et al., 1988, Science 239:491-4). In this approach, a sequencing primer which lies within the amplified sequence is used with double-stranded PCR product or single-stranded template generated by a modified PCR. The sequence determination is performed by conventional procedures with radiolabeled nucleotides or by automatic sequencing procedures with fluorescent tags.

Sequence alterations may occasionally generate fortuitous restriction enzyme recognition sites or may eliminate existing restriction sites. Changes in restriction sites are revealed by the use of appropriate enzyme digestion followed by conventional gel-blot hybridization (Southern, 1975. J. Mol. Biol. 98:503). DNA fragments carrying the site (either normal or mutant) are detected by their reduction in size or increase of corresponding restriction fragment numbers. Genomic DNA samples may also be amplified by PCR prior to treatment with the appropriate restriction enzyme; fragments of different sizes are then visualized under UV light in the presence of ethidium bromide after gel electrophoresis.

Genetic testing based on DNA sequence differences may be achieved by detection of alteration in electrophoretic mobility of DNA fragments in gels with or without denaturing reagent. Small sequence deletions and insertions can be visualized by high-resolution gel electrophoresis. For example, a PCR product with small deletions is clearly distinguishable from a normal sequence on an 8% non-denaturing polyacrylamide gel (WO 91/10734; Nagamine et al., 1989, Am. J. Hum. Genet. 45:337-9). DNA fragments of different sequence compositions may be distinguished on denaturing formamide gradient gels in which the mobilities of different DNA fragments are retarded in the gel at different positions according to their specific “partial-melting” temperatures (Myers et al., 1985. Science 230:1242). Alternatively, a method of detecting a mutation comprising a single base substitution or other small change could be based on differential primer length in a PCR. For example, an invariant primer could be used in addition to a primer specific for a mutation. The PCR products of the normal and mutant genes can then be differentially detected in acrylamide gels.

In addition to conventional gel-electrophoresis and blot-hybridization methods, DNA fragments may also be visualized by methods where the individual DNA samples are not immobilized on membranes. The probe and target sequences may be both in solution, or the probe sequence may be immobilized (Saiki et al., 1989, Proc. Nat. Acad. Sci. USA 86:62304). A variety of detection methods, such as autoradiography involving radioisotopes, direct detection of radioactive decay (in the presence or absence of scintillant), spectrophotometry involving calorigenic reactions and fluorometry involved fluorogenic reactions, may be used to identify specific individual genotypes.

If more than one mutation is frequently encountered in the p52 or p75 gene a system capable of detecting such multiple mutations would be desirable. For example, a PCR with multiple, specific oligonucleotide primers and hybridization probes may be used to identify all possible mutations at the same time (Chamberlain et al., 1988, Nucl. Acids Res. 16:1141-55). The procedure may involve immobilized sequence-specific oligonucleotides probes (Saiki et al. 1989, Proc. Nat. Acad. Sci. USA 86:6230-4).

### EXAMPLE 23

### Quantitation of p52 and p75 Proteins

An alternative method of diagnosing a p52 and/or p75 gene deletion or mutation is to quantitate the level of p52 and/or p75 proteins in the cells of a subject. This diagnostic tool would be useful for detecting reduced levels of the p52 or p75 protein which result from, for example, mutations in the promoter regions of the p52 or p75 gene or mutations within the coding region of the gene which produced truncated, non-functional polypeptides, as well as from deletions of the entire p52 or p75 gene. These diagnostic methods, in addition to those described in EXAMPLE 22, provide an enhanced ability to diagnose susceptibility to diseases caused by mutation or deletion of these genes.

The determination of reduced p52 or p75 protein levels would be an alternative or supplemental approach to the direct determination of p52 or p75 gene deletion or mutation status by the methods outlined above in EXAMPLE 22. The availability of antibodies specific to the p52 or p75 protein (for example those described in EXAMPLES 3 and 21) will facilitate the quantitation of cellular p52 or p75 protein by one of a number of immunoassay methods which are well known in the art and are presented in Harlow and Lane (Antibodies, A Laboratory Manual, Cold Spring Harbor Laboratory, New York. 1988).

Such assays permit both the detection of p52 and p75 proteins in a biological sample and the quantitation of such proteins. Typical methods involve: providing a biological sample of the subject in which the sample contains cellular proteins, and providing an immunoassay for quantitating the level of p52 or p75 protein in the biological sample. This can be achieved by combining the biological sample with a p52 and/or p75 specific binding agent, such as an anti-p52 or anti-p75 antibody (such as monoclonal or polyclonal antibodies), so that complexes form between the binding agent and the p52 and/or p75 protein present in the sample, and then detecting or quantitating such complexes.

In particular forms, these assays may be performed with the p52 and/or p75 specific binding agent immobilized on a support surface, such as in the wells of a microtiter plate or on a column. The biological sample is then introduced onto the support surface and allowed to interact with the specific binding agent so as to form complexes. Excess biological sample is then removed by washing, and the complexes are detected with a reagent, such as a second anti-p52 or -p75 protein antibody that is conjugated with a detectable marker.

In an alternative assay, the cellular proteins are isolated and subjected to SDS-PAGE followed by Western blotting, for example as described in EXAMPLE 19. After resolving the proteins, the proteins are transferred to a membrane, which is probed with specific binding agents that recognize p52 and/or p75. The proteins are detected, for example with HRP-conjugated secondary antibodies, and quantitated.

In yet another assay, the level of p52 and p75 protein in cells is analyzed using microscopy. Using specific binding agents which recognize p52 and/or p75, samples can be analyzed for the presence of p52 and/or p75 proteins. For example, frozen biopsied tissue sections are thawed at room temperature and fixed with acetone at −200° C. for 5 minutes. Slides are washed twice in cold PBS for 5 minutes each, then air-dried. Sections are covered with 20-30 μl of antibody solution (15-45 μg/ml) (diluted in PBS, 2% BSA at 15-50 μg/ml) and incubated at room temperature in humidified chamber for 30 min. Slides are washed three times with cold PBS 5 minutes each, allowed to air-dry briefly (5 minutes) before applying 20-30 μl of the second antibody solution (diluted in PBS, 2% BSA at 15-50 μg/ml) and incubated at room temperature in humidified chamber for 30 minutes. The label on the second antibody may contain a fluorescent probe, enzyme, radiolabel, biotin, or other detectable marker. The slides are washed three times with cold PBS 5 minutes each then quickly dipped in distilled water, air-dried, and mounted with PBS containing 30% glycerol. Slides can be stored at 4° C. prior to viewing.

For samples prepared for electron microscopy (versus light microscopy), the second antibody is conjugated to gold particles. Tissue is fixed and embedded with epoxy plastics, then cut into very thin sections (˜1-2 μm). The specimen is then applied to a metal grid, which is then incubated in the primary anti-p52 or anti-p75 antibody, washed in a buffer containing BSA, then incubated in a secondary antibody conjugated to gold particles (usually 5-20 run). These gold particles are visualized using electron microscopy methods.

For the purposes of quantitating the p52 and p75 proteins, a biological sample of the subject, which sample includes cellular proteins, is required. Such a biological sample may be obtained from body cells, such as those present in which expression of the protein has been detected. As shown in FIG. 5, for example, p52 and p75 could be analyzed in cells isolated from the testis, thymus or brain, but its expression in peripheral blood leukocytes is clearly the most accessible and convenient source from which specimens can be obtained. Specimens can be obtained from peripheral blood, urine, saliva, tissue biopsy, amniocentesis samples, surgical specimens, fine needle aspirates or other breast biopsies, and autopsy material, particularly breast/mammary cancer cells. Quantitation of p52 and p75 proteins would be made by immunoassay and compared to levels of the protein found in non-p52 and non-p75 expressing human cells (i.e. lung and liver) or to the level of p52 or p75 in healthy cells (cells of the same origin that are not neoplastic). A significant (for example 50% or greater) reduction in the amount of p52 and/or p75 protein in the cells of a subject compared to the amount of p52 and/or p75 protein found in non-p52 and/or p75 expressing human cells or that found in normal human cells, would be taken as an indication that the subject may have deletions or mutations in the p52 or p75 gene locus.

### EXAMPLE 24

### Two Step Assay to Detect the Presence of p52 or p75 Gene in a Sample

Breast or other tissue sample from a subject is processed according to the method disclosed by Antonarakis, et al. (New Eng. J. Med. 313:842-848, 1985), separated through a 1% agarose gel and transferred to a nylon membrane for Southern blot analysis. Membranes are UV cross linked at 150 mJ using a GS Gene Linker (Bio-Rad). A p52 or p75 probe (such as those shown in FIG. 5C) is subcloned into pTZ18U. The phagemids are transformed into E. coli MV 1190 infected with M13KO7 helper phage (Bio-Rad, Richmond. Calif.). Single stranded DNA is isolated according to standard procedures (Sambrook, et al. Molecular Cloning: A Laboratory Manual, Cold Spring Harbor, N.Y., 1989).

Blots are prehybridized for 15-30 minutes at 65° C. in 7% sodium dodecyl sulfate (SDS) in 0.5 M NaPO₄. The methods follow those described by Nguyen, et al. (BioTechniques 13:116-123, 1992). The blots are hybridized overnight at 65° C. in 7% SDS, 0.5 M NaPO₄ with 25-50 ng/ml single stranded probe DNA. Post-hybridization washes consist of two 30 minute washes in 5% SDS, 40 mM NaPO₄ at 65° C., followed by two 30-minute washes in 1% SDS, 40 mM NaPO₄ at 65° C.

Next the blots are rinsed with phosphate buffered saline (pH 6.8) for 5 minutes at room temperature (RT) and incubated with 0.2% casein in PBS for 5 minutes. The blots are then preincubated for 5-10 minutes in a shaking water bath at 45° C. with hybridization buffer consisting of 6 M urea, 0.3 M NaCl, and 5× Denhardt's solution (see Sambrook, et al., Molecular Cloning: A Laboratory Manual, Cold Spring Harbor, N.Y., 1989). The buffer is removed and replaced with 50-75 μl/cm2 fresh hybridization buffer plus 2.5 nM of the covalently cross-linked oligonucleotide sequence complementary to the universal primer site (UP-AP, Bio-Rad). The blots are hybridized for 20-30 minutes at 45° C. and post hybridization washes are incubated at 45° C. as two 10 minute washes in 6 M urea, 1× standard saline citrate (SSC), 0.1% SDS and one 10 minute wash in 1×SSC, 0.1% Triton>X-100. The blots are rinsed for 10 minutes at RT with 1×SSC.

Blots are incubated for 10 minutes at RT with shaking in the substrate buffer consisting of 0.1 M diethanolamine, 1 mM MgCl₂, 0.02% sodium azide, pH 10.0. Individual blots are placed in heat sealable bags with substrate buffer and 0.2 mM AMPPD (3-(2′-spiroadamantane)-4-methoxy-4-(3′-phosphoryloxy)phenyl-1,2-dioxetane, disodium salt, Bio-Rad). After a 20 minute incubation at RT with shaking, excess AMPPD solution is removed and the blot is exposed to X-ray film overnight. Positive bands indicate the presence of the p52 and/or p75 gene. Patient samples which show no hybridizing bands lack the p52 and/or p75 gene, indicating the possibility of ongoing cancer, or an enhanced susceptibility to developing cancer in the future.

### EXAMPLE 25

### Gene Therapy

A new gene therapy approach for patients suffering from p52 or p75 gene deletions or mutations is now made possible by the present invention. Essentially, cells, such as breast cells may be removed from a patient having deletions or mutations of the p52 or p75 gene, and then transfected with an expression vector containing the p52 or p75 cDNA. These transfected cells will thereby produce functional p52 or p75 protein and can be reintroduced into the patient. In addition to breast cells, colorectal, prostate, or other cells may be used, depending on the tissue of interest.

The scientific and medical procedures required for human cell transfection are now routine procedures. The provision herein of p52 or p75 cDNAs now allows the development of human gene therapy based upon these procedures. Immunotherapy of melanoma patients using genetically engineered tumor-infiltrating lymphocytes (TILs) has been reported by Rosenberg et al. (N. Engl. J. Med. 323:570-8, 1990). In that study, a retrovirus vector was used to introduce a gene for neomycin resistance into TILs. A similar approach may be used to introduce the p52 or p75 cDNA into subjects affected by p52 or p75 deletions or mutations.

In some embodiments, the present invention relates to a method of treating tumors which underexpress p52 and/or p75. These methods may be accomplished by introducing a gene coding for p52 (or variant thereof) into the subject. A general strategy for transferring genes into donor cells is disclosed in U.S. Pat. No. 5,529,774, which is incorporated by reference. Generally, a gene encoding a protein having therapeutically desired effects is cloned into a viral expression vector, and that vector is then introduced into the target organism. The virus infects the cells, and produces the protein sequence in viva, where it has its desired therapeutic effect. See, for example, Zabner et al. (Cell 75:207-16, 1993).

In some of the foregoing examples, it may only be necessary to introduce the genetic or protein elements into certain cells or tissues. For example, in the case of benign nevi and psoriasis, introducing them into only the skin may be sufficient. However, in some instances (i.e. tumors and polycythemia inflammatory fibrosis), it may be more therapeutically effective and simple to treat all of the patients cells, or more broadly disseminate the vector, for example by intravascular administration.

The nucleic acid sequence encoding at least one therapeutic agent is under the control of a suitable promoter. Suitable promoters which may be employed include, but are not limited to, the gene's native promoter, retroviral LTR promoter, or adenoviral promoters, such as the adenoviral major late promoter, the cytomegalovirus (CMV) promoter; the Rous Sarcoma Virus (RSV) promoter; inducible promoters, such as the MMTV promoter: the metallothionein promoter; heat shock promoters; the albumin promoter; the histone promoter; the α-actin promoter; TK promoters; B19 parvovirus promoters; and the ApoA1 promoter. However the scope of the present invention is not limited to specific foreign genes or promoters.

The recombinant nucleic acid can be administered to the subject by any method which allows the recombinant nucleic acid to reach the appropriate cells. These methods include injection, infusion, deposition, implantation, or topical administration. Injections can be intradermal or subcutaneous. The recombinant nucleic acid can be delivered as part of a viral vector, such as avipox viruses, recombinant vaccinia virus, replication-deficient adenovirus strains or poliovirus, or as a non-infectious form such as naked DNA or liposome encapsulated DNA.

### EXAMPLE 26

### Viral Vectors for Gene Therapy

Adenoviral vectors may include essentially the complete adenoviral genome (Shenk et al., Curr. Top. Microbiol. Immunol. 111:1-39, 1984). Alternatively, the adenoviral vector may be a modified adenoviral vector in which at least a portion of the adenoviral genome has been deleted. In one embodiment, the vector includes an adenoviral 5, ITR; an adenoviral 3′ ITR; an adenoviral encapsidation signal; a DNA sequence encoding a therapeutic agent; and a promoter for expressing the DNA sequence encoding a therapeutic agent. The vector is free of at least the majority of adenoviral E1 and E3 DNA sequences, but is not necessarily free of all of the E2 and E4 DNA sequences, and DNA sequences encoding adenoviral proteins transcribed by the adenoviral major late promoter. In another embodiment, the vector may be an adeno-associated virus (AAV) such as described in U.S. Pat. No. 4,797,368 (Carter et al.) and in McLaughlin et al. (J. Virol. 62:1963-73, 1988) and AAV type 4 (Chiorini et al. J. Virol. 71:6823-33, 1997) and AAV type 5 (Chiorini et al. J. Virol. 73:1309-19, 1999)

Such a vector may be constructed according to standard techniques, using a shuttle plasmid which contains, beginning at the 5′ end, an adenoviral 5′ ITR an adenoviral encapsidation signal, and an E1a enhancer sequence; a promoter (which may be an adenoviral promoter or a foreign promoter): a tripartite leader sequence, a multiple cloning site (which may be as herein described); a poly A signal; and a DNA segment which corresponds to a segment of the adenoviral genome. The DNA segment serves as a substrate for homologous recombination with a modified or mutated adenovirus, and may encompass, for example, a segment of the adenovirus 5′ genome no longer than from base 3329 to base 6246. The plasmid may also include a selectable marker and an origin of replication. The origin of replication may be a bacterial origin of replication. A desired DNA sequence encoding a therapeutic agent may be inserted into the multiple cloning site of the plasmid.

The plasmid may be used to produce an adenoviral vector by homologous recombination with a modified or mutated adenovirus in which at least the majority of the E1 and E3 adenoviral DNA sequences have been deleted. Homologous recombination may be effected through co-transfection of the plasmid vector and the modified adenovirus into a helper cell line, such as 293 cells, by CaPO₄ precipitation. The homologous recombination produces a recombinant adenoviral vector which includes DNA sequences derived from the shuttle plasmid between the Not I site and the homologous recombination fragment, and DNA derived from the E1 and E3 deleted adenovirus between the homologous recombination fragment and the 3′ ITR.

In one embodiment, the adenovirus may be constructed by using a yeast artificial chromosome (or YAC) containing an adenoviral genome according to the method described in Ketner et al. (Proc. Natl. Acad. Sci. USA, 91:6186-90, 1994), in conjunction with the teachings contained herein. In this embodiment, the adenovirus yeast artificial chromosome is produced by homologous recombination in vivo between adenoviral DNA and yeast artificial chromosome plasmid vectors carrying segments of the adenoviral left and right genomic termini. A DNA sequence encoding a therapeutic agent then may be cloned into the adenoviral DNA. The modified adenoviral genome then is excised from the adenovirus yeast artificial chromosome in order to be used to generate adenoviral vector particles as hereinabove described.

The adenoviral particles are administered in an amount effective to produce a therapeutic effect in a subject. The exact dosage of adenoviral particles to be administered is dependent upon a variety of factors, including the age, weight, and sex of the subject to be treated, and the nature and extent of the disease or disorder to be treated. The adenoviral particles may be administered as part of a preparation having a titer of adenoviral particles of at least 1×10¹⁰ pfu/ml, and in general not exceeding 2×10¹¹ pfu/ml. The adenoviral particles may be administered in combination with a pharmaceutically acceptable carrier in a volume up to 10 ml. The pharmaceutically acceptable carrier may be, for example, a liquid carrier such as a saline solution, protamine sulfate (Elkins-Sinn, Inc., Cherry Hill, N.J.), Polybrene (Sigma Chemical), agents described in the DEFINITION section above, or those agents described in EXAMPLE 33.

In another embodiment, the viral vector is a retroviral vector. Retroviruses have been considered for experiments in gene therapy because they have a high efficiency of infection and stable integration and expression (Orkin et al. 1988, Prog. Med. Genet. 7:13042). The full length p52 or p75 gene or cDNA can be cloned into a retroviral vector and driven from either its endogenous promoter or from the retroviral LTR (long terminal repeat). Examples of retroviral vectors which may be employed include, but are not limited to, Moloney Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus. The vector is generally a replication defective retrovirus particle.

Retroviral vectors are useful as agents to effect retroviral-mediated gene transfer into eukaryotic cells. Retroviral vectors are generally constructed such that the majority of sequences coding for the structural genes of the virus are deleted and replaced by the gene(s) of interest. Most often, the structural genes (i.e., gag, pol, and env), are removed from the retroviral backbone using genetic engineering techniques known in the art. This may include digestion with the appropriate restriction endonuclease or, in some instances, with Bal 31 exonuclease to generate fragments containing appropriate portions of the packaging signal.

Other viral transfection systems may also be utilized for this type of approach, including Vaccinia virus (Moss et al., 1987, Annu. Rev. Immunol. 5:305-24), Bovine Papilloma virus (Rasmussen et al., 1987, Methods Enzymol. 139:642-54) or members of the herpes virus group such as Epstein-Barr virus (Margolskee et al., 1988, Mol. Cell. Biol. 8:283747). Recent developments in gene therapy techniques include the use of RNA-DNA hybrid oligonucleotides, as described by Cole-Strauss, et al. (Science 273:1386-9, 1996). This technique can allow for site-specific integration of cloned sequences, permitting accurately targeted gene replacement.

New genes may be incorporated into proviral backbones in several general ways. In the most straightforward constructions, the structural genes of the retrovirus are replaced by a single gene which then is transcribed under the control of the viral regulatory sequences within the long terminal repeat (LTR). Retroviral vectors have also been constructed which can introduce more than one gene into target cells. Usually, in such vectors one gene is under the regulatory control of the viral LTR, while the second gene is expressed either off a spliced message or is under the regulation of its own, internal promoter. Alternatively, two genes may be expressed from a single promoter by the use of an Internal Ribosome Entry Site.

### EXAMPLE 27

### Cloning of p52 and p75 Genomic DNA

This example describes methods for cloning p52 and p75 genomic DNA from any species. Such methods are known to those skilled in the art, and are described in Sambrook et al. (Molecular Cloning: A Laboratory Manual, Cold Spring Harbor Laboratory Press, New York.

1989. Herein incorporated by reference). Briefly, p52 and/or p75 cDNA (full length or fragments thereof, for example SEQ ID NOs 1 and 3) is radiolabeled with rediprime II (Amersham Pharmacia Biotech, Piscataway, N.J.) as instructed by the manufacturer. This radiolabeled cDNA is used to screen a bacteriophage lambda gt11 genomic library. Genomic DNA of the resulting positive clones is isolated, purified and digested with appropriate restriction enzymes. Digested DNA is separated by agarose gel electrophoresis and blotted onto a nylon membrane. A Southern-Blot is performed using radioactive cDNA of p52 and/or p75 to identify the exons. Bands that hybridized with the cDNA are isolated from the gel and sequenced. The resulting DNA sequence is analyzed by specific computer programs to identify the promoter region and exon/intron donor/acceptor sites.

### EXAMPLE 28

### Sequence Variants of p52 and p75

The nucleotide sequence of the p52 and p75 cDNAs (SEQ ID NOs 3 and 1, respectively) and the amino acid sequence of the p52 and p75 proteins (SEQ ID NOs 4 and 2 respectively) which are encoded by the cDNAs, respectively, are shown in FIGS. 1-3. Having presented the nucleotide sequence of the p52 and p75 cDNAs and the amino acid sequence of these proteins, this invention now also facilitates the creation of DNA molecules, and thereby proteins, which are derived from those disclosed but which vary in their precise nucleotide or amino acid sequence from those disclosed. Such variants may be obtained through a combination of standard molecular biology laboratory techniques and the nucleotide sequence information disclosed by this invention.

Variant DNA molecules include those created by standard DNA mutagenesis techniques, for example, M 13 primer mutagenesis. Details of these techniques are provided in Sambrook et al. (In: Molecular Cloning: A Laboratory Manual, Cold Spring Harbor, N.Y., 1989, Ch. 15). By the use of such techniques, variants may be created which differ in minor ways from those disclosed. DNA molecules and nucleotide sequences which are derivatives of those specifically disclosed herein and which differ from those disclosed by the deletion, addition or substitution of nucleotides while still encoding a protein which possesses the functional characteristics of the p52 and p75 proteins are comprehended by this invention. Also within the scope of this invention are small DNA molecules which are derived from the disclosed DNA molecules. Such small DNA molecules include oligonucleotides suitable for use as hybridization probes or polymerase chain reaction (PCR) primers. As such, these small DNA molecules will comprise at least a segment of the p52 or p75 cDNA molecules or the p52 or p75 gene and, for the purposes of PCR, will comprise at least a 15 or a 20-50 nucleotide sequence of the p52 and p75 cDNAs (SEQ ID NOs 3 and 1 respectively) or the p52 and p75 genes (i.e., at least 20-50 consecutive nucleotides of the p52 or p75 cDNA or gene sequences). DNA molecules and nucleotide sequences which are derived from the disclosed DNA molecules as described above may also be defined as DNA sequences which hybridize under stringent conditions to the DNA sequences disclosed, or fragments thereof.

Hybridization conditions resulting in particular degrees of stringency will vary depending upon the nature of the hybridization method of choice and the composition and length of the hybridizing DNA used. Generally, the temperature of hybridization and the ionic strength (especially the Na⁺ concentration) of the hybridization buffer will determine the stringency of hybridization. Calculations regarding hybridization conditions required for attaining particular degrees of stringency are discussed by Sambrook et al. (In: Molecular Cloning: A Laboratory Manual, Cold Spring Harbor, N.Y., 1989 ch. 9 and 11), herein incorporated by reference. By way of illustration only, a hybridization experiment may be performed by hybridization of a DNA molecule (for example, a deviation of the p52, or p75 cDNA) to a target DNA molecule (for example, the p52 or p75 cDNA) which has been electrophoresed in an agarose gel and transferred to a nitrocellulose membrane by Southern blotting (Southern, J. Mol. Biol. 98:503, 1975), a technique well known in the art and described in Sambrook et al. (Molecular Cloning: A Laboratory Manual, Cold Spring Harbor, N.Y., 1989). Hybridization with a target probe labeled with [³²P]-dCTP is generally carried out in a solution of high ionic strength such as 6×SSC at a temperature that is 20-25° C. below the melting temperature, Tₘ, described below. For such Southern hybridization experiments where the target DNA molecule on the Southern blot contains 0.10 ng of DNA or more, hybridization is typically carried out for 6-8 hours using 1-2 ng/ml radiolabeled probe (of specific activity equal to 10⁹ CPM/μg or greater). Following hybridization, the nitrocellulose filter is washed to remove background hybridization. The washing conditions should be as stringent as possible to remove background hybridization but to retain a specific hybridization signal. The term Tₘ represents the temperature above which, under the prevailing ionic conditions, the radiolabeled probe molecule will not hybridize to its target DNA molecule. The Tₘ of such a hybrid molecule may be estimated from the following equation (Bolton and McCarthy, Proc. Natl. Acad. Sci. USA 48:1390, 1962): Tₘ=81.5° C.-16.6(log₁₀[Na⁺])+0.41(% G+C)−0.63(% formamide)−(600/1); where I=the length of the hybrid in base pairs.

This equation is valid for concentrations of Na⁺ in the range of 0.01 M to 0.4 M, and it is less accurate for calculations of Tₘ in solutions of higher [Na⁺]. The equation is also primarily valid for DNAs whose G+C content is in the range of 30% to 75%, and it applies to hybrids greater than 100 nucleotides in length (the behavior of oligonucleotide probes is described in detail in Ch. 11 of Sambrook et al. (Molecular Cloning: A Laboratory Manual, Cold Spring Harbor, N.Y., 1989).

Thus, by way of example, for a 150 base pair DNA probe derived from the open reading frame of the p52 or p75 cDNA (with a hypothetical % GC=45%), a calculation of hybridization conditions required to give particular stringencies may be made as follows. For this example, it is assumed that the filter will be washed in 0.3×SSC solution following hybridization, thereby: [Na⁺]=0.045M: % GC=45%; Formamide concentration=0; 1=150 base pairs; Tₘ=81.5-16.6(log₁₀[Na⁺]+(0.41×45)−(600/150); and so Tₘ=74.4° C.

The Tₘ of double-stranded DNA decreases by 1-1.5° C. with every 1% decrease in homology (Bonner et al., J. Mol. Biol. 81:123, 1973). Therefore, for this given example, washing the filter in 0.3×SSC at 59.4-64.4° C. will produce a stringency of hybridization equivalent to 90%; that is, DNA molecules with more than 10% sequence variation relative to the target DLC-I cDNA will not hybridize. Alternatively, washing the hybridized filter in 0.3×SSC at a temperature of 65.4-68.4° C. will yield a hybridization stringency of 94%; that is, DNA molecules with more than 6% sequence variation relative to the target p52 or p75 cDNA molecule will not hybridize. The above example is given entirely by way of theoretical illustration. One skilled in the art will appreciate that other hybridization techniques may be utilized and that variations in experimental conditions will necessitate alternative calculations for stringency.

In particular embodiments of the present invention, stringent conditions may be defined as those under which DNA molecules with more than 25%, 15%, 10%, 6% or 2% sequence variation (also termed “mismatch”) will not hybridize.

The degeneracy of the genetic code further widens the scope of the present invention as it enables major variations in the nucleotide sequence of a DNA molecule while maintaining the amino acid sequence of the encoded protein. For example, the thirteenth amino acid residue of the p52 protein is alanine. This is encoded in the p52 cDNA by the nucleotide codon triplet GCC. Because of the degeneracy of the genetic code, three other nucleotide codon triplets. GCT, GCG and GCA, also code for alanine. Thus, the nucleotide sequence of the p52 cDNA could be changed at this position to any of these three codons without affecting the amino acid composition of the encoded protein or the characteristics of the protein. Based upon the degeneracy of the genetic code, variant DNA molecules may be derived from the cDNA molecules disclosed herein using standard DNA mutagenesis techniques as described above, or by synthesis of DNA sequences. DNA sequences which do not hybridize under stringent conditions to the cDNA sequences disclosed by virtue of sequence variation based on the degeneracy of the genetic code are herein also comprehended by this invention.

The invention also includes DNA sequences that are substantially identical to any of the DNA sequences disclosed herein, where substantially identical means a sequence that has identical nucleotides in at least 75%. 80%, 85%, 90%, 95% or 98% of the aligned sequences.

One skilled in the art will recognize that the DNA mutagenesis techniques described above may be used not only to produce variant DNA molecules, but will also facilitate the production of proteins which differ in certain structural aspects from the p52 or p75 proteins, yet which proteins are clearly derivative of this protein and which maintain the essential characteristics of the p52 or p75 protein. Newly derived proteins may also be selected in order to obtain variations on the characteristic of the p52 or p75 protein, as will be more fully described below. Such derivatives include those with variations in amino acid sequence including minor deletions, additions and substitutions.

While the site for introducing an amino acid sequence variation is predetermined, the mutation per se need not be predetermined. For example, in order to optimize the performance of a mutation at a given site, random mutagenesis may be conducted at the target codon or region and the expressed protein variants screened for the optimal combination of desired activity. Techniques for making substitution mutations at predetermined sites in DNA having a known sequence as described above are well known.

Amino acid substitutions are typically of single residues; insertions usually will be oh the order of about from 1 to 10 amino acid residues; and deletions will range about from 1 to 30 residues. Deletions or insertions can be made in adjacent pairs, i.e., a deletion of 2 residues or insertion of 2 residues. Substitutions, deletions, insertions or any combination thereof may be combined to arrive at a final construct. Obviously, the mutations that are made in the DNA encoding the protein must not place the sequence out of reading frame and for example will not create complementary regions that could produce secondary mRNA structure.

Substitutional variants are those in which at least one residue in the amino acid sequence has been removed and a different residue inserted in its place. Such substitutions generally are made conservatively, as defined above.

Substantial changes in function or immunological identity are made by selecting substitutions that are less conservative than those defined above, i.e., selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g. phenylalanine, is substituted for (or by) one not having a side chain, e.g., glycine.

The effects of these amino acid substitutions or deletions or additions may be assessed for derivatives of the p52 or p75 protein by assays in which DNA molecules encoding the derivative proteins are transfected into p52 or p75 cells using routine procedures. These p52 and p75 would be expressed recombinantly (for example see EXAMPLE 4), purified, and analyzed for their ability to enhance transcription (as compared to normal p52 and p75) and splicing, as described in EXAMPLES 5-7, 10, 14, 17, 18.

### EXAMPLE 29

### Cloning p52 and p75 in Other Species

Having presented the nucleotide sequences of the human p52 and p75 cDNAs (SEQ ID NOs 3 and 1, respectively) and the amino acid sequence of the encoded proteins (SEQ ID NOs 4 and 2, respectively), this invention now also facilitates the identification of DNA molecules, and thereby proteins, which are the p52 and p75 homologs in other species. These other homologs can be derived from those sequences disclosed, but which vary in their precise nucleotide or amino acid sequence from those disclosed. Such variants may be obtained through a combination of standard molecular biology laboratory techniques and the nucleotide and amino acid sequence information disclosed by this invention.

### EXAMPLE 30

### Peptide Modifications

The present invention includes biologically active molecules that mimic the action (mimetics) of the p52 and p75 proteins of the present invention. The invention therefore includes synthetic embodiments of naturally-occurring peptides described herein, as well as analogues (non-peptide organic molecules), derivatives (chemically functionalized peptide molecules obtained starting with the disclosed peptide sequences) and variants (homologs) of these peptides that specifically inhibit the conversion assay reaction. Each peptide ligand of the invention is comprised of a sequence of amino acids, which may be either L- and/or D-amino acids, naturally occurring and otherwise.

Peptides may be modified by a variety of chemical techniques to produce derivatives having essentially the same activity as the unmodified peptides, and optionally having other desirable properties. For example, carboxylic acid groups of the peptide, whether carboxyl-terminal or side chain, may be provided in the form of a salt of a pharmaceutically-acceptable cation or esterified to form a C1-C16 ester, or converted to an amide of formula NR1R2 wherein R1 and R2 are each independently H or C1-C16 alkyl, or combined to form a heterocyclic ring, such as a 5- or 6-membered ring. Amino groups of the peptide, whether amino-terminal or side chain, may be in the form of a pharmaceutically-acceptable acid addition salt, such as the HCl. HBr, acetic, benzoic, toluene sulfonic, maleic, tartaric and other organic salts, or may be modified to C1-C16 alkyl or dialkyl amino or further converted to an amide.

Hydroxyl groups of the peptide side chain may be converted to C1-C16 alkoxy or to a C1-C16 ester using well-recognized techniques. Phenyl and phenolic rings of the peptide side chain may be substituted with one or more halogen atoms, such as fluorine, chlorine, bromine or iodine, or with C1-C16 alkyl, C1-C16 alkoxy, carboxylic acids and esters thereof, or amides of such carboxylic acids. Methylene groups of the peptide sidechains can be extended to homologous C2-C4 alkylenes. Thiols can be protected with any one of a number of well-recognized protecting groups, such as acetamide groups. Those skilled in the art will also recognize methods for introducing cyclic structures into the peptides of this invention to select and provide conformational constraints to the structure that result in enhanced stability. For example, a carboxyl-terminal or amino-terminal cysteine residue can be added to the peptide, so that when oxidized the peptide will contain a disulfide bond, thereby generating a cyclic peptide. Other peptide cyclizing methods include the formation of thioethers and carboxyl- and amino-terminal amides and esters.

In order to maintain an optimally functional peptide, particular peptide variants will differ by only a small number of amino acids from the peptides disclosed in this specification. Such variants may have deletions (for example of 1-3 or more amino acid residues), insertions (for example of 1-3 or more residues), or substitutions that do not interfere with the desired activity of the peptides. Substitutional variants are those in which at least one residue in the amino acid sequence has been removed and a different residue inserted in its place. In particular embodiments, such variants will have amino acid substitutions of single residues, for example 1, 3, 5 or even 10 substitutions in the full length p52 or p75 protein.

Peptidomimetic and organomimetic embodiments are also within the scope of the present invention, whereby the three-dimensional arrangement of the chemical constituents of such peptido- and organomimetics mimic the three-dimensional arrangement of the peptide backbone and component amino acid sidechains in the peptide, resulting in such peptido- and organomimetics of the peptides of this invention having substantial ability to enhance transcription and splicing activity. For computer modeling applications, a pharmacophore is an idealized, three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs”, in Klegerman & Groves, eds., 1993, Pharmaceutical Biotechnology, Interpharm Press: Buffalo Grove, Ill., pp. 165-174 and Principles of Pharmacology (ed. Munson, 1995), chapter 102 for a description of techniques used in CADD. Also included within the scope of the invention are mimetics prepared using such techniques that produce either peptides or conventional organic pharmaceuticals that retain the biological activity of the p52 and/or p75 proteins.

### EXAMPLE 31

### Method for Generating Mimetics

Compounds or other molecules which mimic normal p52 or p75 function, such as compounds which enhance transcription and splicing, can be identified and/or designed. These compounds or molecules are known as mimetics, because they mimic the biological activity of the normal protein.

Crystallography

To identify the amino acids that interact between the transcription factors and p52 or p75, p52 or p75 is co-crystallized in the presence of the transcription factor. In addition, the similar experiments can be conducted to analyze the interaction of p52 and p75 with splicing factors. One method that can be used is the hanging drop method. In this method, a concentrated salt, transcription factor and p52 or p75 protein solution is applied to the underside of a lid of a multiwell dish. A range of concentrations may need to be tested. The lid is placed onto the dish, such that the droplet “hangs” from the lid. As the solvent evaporates, a protein crystal is formed, which can be visualized with a microscope. This crystallized structure is then subjected to X-ray diffraction or NMR analysis which allows for the identification of the amino acid residues that are in contact with one another. The amino acids that contact the transcription factors establish a pharmacophore that can then be used to identify drugs that interact at that same site.

Identification of Drugs

Once these amino acids have been identified, one can screen synthetic drug databases (which can be licensed from several different drug companies), to identify drugs that interact with the same amino acids of p52 or p75 that the transcription or splicing factors interact with. Moreover, structure activity relationships and computer assisted drug design can be performed as described in Remington, The Science and Practice of Pharmacy, Chapter 28.

Designing Synthetic Peptides

In addition, synthetic peptides can be designed from the sequence of the transcription or splicing factor that interacts with p52 or p75. Several different peptides could be generated from this region. This could be done with or without the crystalography data. However, once crystalography data is available, peptides can also be designed that bind better than p52 or p75.

The chimeric peptides may be expressed recombinantly, for example in E. coli. The advantage of the synthetic peptides over the mAbs is that they are smaller, and therefore diffuse easier, and are not as likely to be immunogenic. Standard mutagenesis of such peptides can also be performed to identify variant peptides having even greater enhancement of transcription and splicing.

After synthetic drugs or peptides that bind to transcription and/or splicing factors have been identified, their ability to enhance transcription and splicing, can be tested as described in the above EXAMPLES 5-7,10,14,17,18. Those that are positive would be good candidates for cancer therapies wherein the cancer cells underexpress p52 and/or p75.

### EXAMPLE 32

### Peptide Synthesis and Purification

The peptides provided by the present invention can be chemically synthesized by any of a number of manual or automated methods of synthesis known in the art. For example, solid phase peptide synthesis (SPPS) is carried out on a 0.25 millimole (mmole) scale using an Applied Biosyslems Model 431 A Peptide Synthesizer and using 9-fluorenylmethyloxycarbonyl (Fmoc) amino-terminus protection, coupling with dicyclohexylcarbodiimide/hydroxybenzotriazole or 2-(1H-benzo-triazol-1-yl)-1,1,3,3-tetramethyluronium hexafluorophosphate/hydroxybenzotriazole (HBTU/HOBT), and using p-hydroxymethylphenoxymethylpolystyrene (HMP) or Sasrin resin for carboxyl-terminus acids or Rink amide resin for carboxyl-terminus amides.

Fmoc-derivatized amino acids are prepared from the appropriate precursor amino acids by tritylation and triphenylmethanol in trifluoroacetic acid, followed by Fmoc derivitization as described by Atherton et al. (Solid Phase Peptide Synthesis, IRL Press: Oxford, 1989).

Sasrin resin-bound peptides are cleaved using a solution of 1% TFA in dichloromethane to yield the protected peptide. Where appropriate, protected peptide precursors are cyclized between the amino- and carboxyl-termini by reaction of the amino-terminal free amine and carboxyl-terminal free acid using diphenylphosphorylazide in nascent peptides wherein the amino acid sidechains are protected.

HMP or Rink amide resin-bound products are routinely cleaved and protected sidechain-containing cyclized peptides deprotected using a solution comprised of trifluoroacetic acid (TFA), optionally also comprising water, thioanisole, and ethanedithiol, in ratios of 100:5:5:2.5, for 0.5-3 hours at room temperature.

Crude peptides are purified by preparative high pressure liquid chromatography (HPLC), for example using a Waters Delta-Pak C18 column and gradient elution with 0.1% TFA in water modified with acetonitrile. After column elution, acetonitrile is evaporated from the eluted fractions, which are then lyophilized. The identity of each product so produced and purified may be confirmed by fast atom bombardment mass spectroscopy (FABMS) or electrospray mass spectroscopy (ESMS).

### EXAMPLE 33

### Pharmaceutical Compositions and Modes of Administration

Various delivery systems for administering the combined therapy of the present invention are known, and include e.g., encapsulation in liposomes, microparticles, microcapsules, expression by recombinant cells, receptor-mediated endocytosis (see Wu and Wu, J. Biol. Chem. 1987, 262:4429-32), and construction of a therapeutic nucleic acid as part of a retroviral or other vector. Methods of introduction include, but are not limited to, intradermal, intramuscular, intraperitoneal, intravenous, subcutaneous, intranasal, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, the pharmaceutical compositions may be introduced into the central nervous system by any suitable route, including intraventricular and intrathecal injection; intraventricular injection may be facilitated by an intraventricular catheter, for example, attached to a reservoir, such as an Ommaya reservoir.

In one embodiment, it may be desirable to administer the pharmaceutical compositions of the invention locally to the area in need of treatment, for example, by local infusion during surgery, topical application, e.g., in conjunction with a wound dressing after surgery, by injection, through a catheter, by a suppository or an implant, such as a porous, non-porous, or gelatinous material, including membranes, such as silastic membranes, or fibers. In one embodiment, administration can be by direct injection at the site (or former site) of a malignant tumor or neoplastic or pre-neoplastic tissue.

The use of liposomes as a delivery vehicle is one delivery method of interest. The liposomes fuse with the target site and deliver the contents of the lumen intracellularly. The liposomes are maintained in contact with the target cells for a sufficient time for fusion to occur, using various means to maintain contact, such as isolation and binding agents. Liposomes may be prepared with purified proteins or peptides that mediate fusion of membranes, such as Sendai virus or influenza virus. The lipids may be any useful combination of known liposome forming lipids, including cationic lipids, such as phosphatidylcholine. Other potential lipids include neutral lipids, such as cholesterol, phosphatidyl serine, phosphatidyl glycerol, and the like. For preparing the liposomes, the procedure described by Kato et al. (J. Biol. Chem. 1991, 266:3361) may be used.

The present invention also provides pharmaceutical compositions which include a therapeutically effective amount of the p52 and/or p75 proteins, RNA or DNAs, alone or with a pharmaceutially acceptable carrier.

Delivery Systems

Such carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, and magnesium carbonate.

In a particular embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule, indicating the quantity of active agent. Where the composition is to be administered by infusion, it can be dispensed with an infusion bottle containing sterile pharmaceutical grade water or saline.

The compositions can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, and procaine. The amount of the active agent that will be effective in the treatment of a particular disorder or condition will depend on the nature of the disorder or condition, and can be determined by standard clinical techniques. In addition, in vitro assays may optionally be employed to help identify optimal dosage ranges, and in vivo dosages can be those sufficient to achieve tissue concentrations at a site of action which are at least as great as those determined in vitro. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration.

The pharmaceutical compositions or methods of treatment may be administered in combination with other therapeutic treatments, such as other antineoplastic or antitumorigenic therapies.

Administration of Nucleic Acid Molecules

In an embodiment in which a p52 and/or p75 nucleic acid is employed for gene therapy, the analog is delivered intracellularly (e.g., by expression from a nucleic acid vector or by receptor-mediated mechanisms). In an embodiment where the therapeutic molecule is a nucleic acid, administration may be achieved by an appropriate nucleic acid expression vector which is administered so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Nail. Acad. Sci. USA 1991, 88:1864-8), etc. Alternatively, the nucleic acid can be introduced intracellularly and incorporated within host cell DNA for expression, by homologous recombination.

The vector pCDNA, is an example of a method of introducing the foreign cDNA into a cell under the control of a strong viral promoter (CMV) to drive the expression. However, other vectors can be used (see EXAMPLE 26). Other retroviral vectors (such as pRETRO-ON, Clontech), also use this promoter but have the advantages of entering cells without any transfection aid, integrating into the genome of target cells ONLY when the target cell is dividing (as cancer cells do, especially during first remissions after chemotherapy) and they are regulated. It is also possible to turn on the expression of the p52 and/or p75 nucleic acids by administering tetracycline when these plasmids are used. Hence these plasmids can be allowed to transfect the cells, then administer a course of tetracycline with a course of chemotherapy to achieve better cytotoxicity.

Other plasmid vectors, such as pMAM-neo (also from Clontech) or pMSG (Pharmacia) use the MMTV-LTR promoter (which can be regulated with steroids) or the SVIO late promoter (pSVL, Pharmacia) or metallothionein-responsive promoter (pBPV, Pharmacia) and other viral vectors, including retroviruses. Examples of other viral vectors include adenovirus, AAV (adeno-associated virus), recombinant HSV, poxviruses (vaccinia) and recombinant lentivirus (such as HIV). All these vectors achieve the basic goal of delivering into the target cell the cDNA sequence and control elements needed for transcription. The present invention includes all forms of nucleic acid delivery, including synthetic oligos, naked DNA, plasmid and viral, integrated into the genome or not.

Administration of Antibodies

In an embodiment where the therapeutic molecule is an antibody, specifically an antibody that recognizes both p52 and p75 or that recognizes p52 or p75 proteins, administration may be achieved by direct injection, or by use of microparticle bombardment (e.g., a gene gun; Biolistic, Dupont), or coating with lipids or cell-surface receptors or transfecting agents. Similar methods can be used to administer p52 and p75 proteins, of fragments thereof.

The present invention also provides pharmaceutical compositions which include a therapeutically effective amount of the antibody, and a pharmaceutically acceptable carrier or excipient.

### EXAMPLE 34

### Transgenic Plants and Animals

The creation of transgenic plants and animals which express p52 and/or p75 can be made by techniques known in the art, for example those disclosed in U.S. Pat. Nos. 5,574,206; 5,723,719; 5,175,383; 5,824,838; 5,811,633; 5,620,881; and 5,767,337, which are incorporated by reference.

Animals which do not express p52 or p75 in their cells can be prepared to further investigate the role of p52 an p75 on transcription, splicing and tumorigenesis. Methods for generating transgenic mice are described in Gene Targeting, A. L. Joyuner ed., Oxford University Press, 1995 and Watson, J. D. et al., Recombinant DNA 2nd Ed., W.H. Freeman and Co. New York, 1992, Chapter 14. To generate transgenic mice containing a functional deletion of the p52 and/or p75 gene, genomic fragments can be used as short arm and long arm. Between long arm and short arm, the neo gene is introduced, generating a the knock-out vector.

Using standard transgenic mouse technology, the knock-out vector can be used to generate p52 and/or p75 knock-out mice by homologous recombination. The knock-out vector is introduced into embryonic stem cells (ES cells) by standard methods which may include transfection, retroviral infection or electroporation (also see EXAMPLE 20). The transfected ES cells expressing the knock-out vector will grow in medium containing the antibiotic G418. The neomycin resistant ES cells will be microinjected into mouse embryos (blastocysts), which are implanted into the uterus of pseudopregnant mice. The litter will be screened for chimeric mice by observing their coat color and by screening for the presence of the transgene by PCR on tail snippets. Chimeric mice are ones in which the injected ES cells developed into the germ line, thereby allowing transmission of the gene to their offspring. The resulting heterozygotic mice are interbred to generate a homozygous line of transgenic mice functionally deleted for p52 and/or p75. These homozygous mice will then be screened phenotypically, for example, their predisposition to developing diseases such as cancer.

Alternatively, the method of Kim et al. (Nature, 383:542-6, 1996) can be used. Briefly, a targeting vector is constructed by replacing a fragment containing p52 or p75 exons with the neo-resistance cassette in the vector pPNT. The herpes simplex virus thymidine kinase (HSV-TK) gene is inserted downstream of the long arm. The linearized targeting vector is transfected into embryonic stem cell lines E14 and CJ-7. G418 and gancyclovir-resistant clones are screened for homologous recombination by PCR and Western blotting. Correctly targeted ES clones are obtained (see above for screening method) and injected into C5BL/6 blastocysts. Heterozygous offspring of the germline-transmitting chimeras are interbred to obtain homozygous mice.

### EXAMPLE 35

### DT40 Knock-Out Cells

This example provides a method that can be used to determine the function of p52 and/or p75 in vivo, by functionally deleting p52 and/or p75 in DT40 cells.

Briefly, using the method described by Wang et al. (Gene. Devel. 10:2588-99, 1996), after cloning the chicken p52 and p75 genomic DNAs using the methods described in EXAMPLE 27, bacterial hygromycin or neomycin-resistance genes, each driven by the chicken β-actin promotor, are inserted into one of the p52 or p75 exons. Plasmids are constructed using standard subcloning procedures generating the constructs Neo-p52, Neo-75, Hygro-p52 and Hygro-p75. The Hygro-constructs are transfected into the chicken B-cell line DT-40.

DT40 cells are maintained in RPMI 1640 medium supplemented with 10% fetal bovine serum and 1% chicken serum at 37° C. at 5% CO₂. For each transfection, approximately 10⁷ cells are suspended in 0.5 ml PBS containing 30 μg linearized plasmid and electroporated with a Gene Pulser apparatus (BioRad) at 550 V and 25° F. Following electroporation, cells are incubated in fresh medium lacking drugs for 24 hours. Cells are then resuspended in fresh medium containing 1.5 mg/ml hygromycin (Calbiochem). After 7-10 days, hygromycin-resistant colonies will be observed and isolated. Positive clones are screened for homologous recombination by Southern blotting, for example using a radiolabeled p52 or p75 probe, such as those shown in FIG. 5.

If the DT40 cells can survive with only one allele of p52 and/or p75, a second round of gene targeting will be used to disrupt the second p52 and/or p75 allele. To accomplish this, one of the heterozygous clones isolated above will be transfected with Neo-p52 and/or Neo-p75, and selected in medium containing both hygromycin and G418 (2 mg/ml, Gibco, BRL, Rockville, Md.). Resulting clones that are resistant to both G418 and hygromycin will be screened by Southern blot as described above, to determine if homologous recombination occurred. If homologous recombination is not observed, this indicates that p52 and/or p75 is an essential gene in DT40 cells.

The resulting recombinant DT40 cells can be used to further investigate the role of p52 and p75 on transcription and splicing, using the methods provided in EXAMPLES 5-7, 10, 14, 17, and 18. In addition, the p52 and/or p75-knock-out DT40 cells can be used to study the effect of p52 and/or p75 on cell growth and the expression of other genes.

Having illustrated and described the principles of isolating the human p52 or p75 cDNA and its protein and modes of use of these biological molecules, it should be apparent to one skilled in the art that the invention can be modified in arrangement and detail without departing from such principles. In view of the many possible embodiments to which the principles of my invention may be applied, it should be recognized that the illustrated embodiments are only preferred examples of the invention and should not be taken as a limitation on the scope of the invention. Rather, the scope of the invention is in accord with the following claims. I therefore claim as my invention all that comes within the scope and spirit of these claims.

1. A purified polypeptide comprising SEQ ID NO: 5.

2. The polypeptide of claim 1, wherein the polypeptide comprises SEQ ID NO: 2

3. The polypeptide of claim 1, wherein the sequence comprises SEQ ID NO: 4.

4. A The purified polypeptide of claim 2 wherein the polypeptide consists essentially of SEQ ID NO 2.

5. A The purified polypeptide of claim 3, wherein the polypeptide consists essentially of SEQ ID NO: 4.

6. A The purified polypeptide of claim 2, wherein the polypeptide consists of SEQ ID NO: 2.

7. A The purified polypeptide of claim 3, wherein the polypeptide consists of SEQ ID NO: 4.

8. A purified polypeptide having an activity of p52 or p75, and which includes an amino acid sequence shown in SEQ ID NO:s 2 or 4.

9. A purified polypeptide having an activity of p52 or p75, and which includes an amino acid sequence shown in SEQ ID NO: 8.

10. The purified polypeptide of claim 9, wherein the polypeptide has an activity of p52, and which includes an amino acid sequence shown in SEQ ID NO: 8.

11. A purified polypeptide that acts as a general coactivator of transcription in an in vitro transcription assay, and specifically interacts with ASF/SF2 to elevate proximal small t 5′ splice site selection of SV40 early pre-mRNA in the presence of HeLa cell nuclear extract or HeLa cell S100 extract and ASF/SF2.

12. A purified polypeptide according to claim of 11, wherein the polypeptide can enhance transcription of transcriptional activators containing an acidic activation domain.

13. A purified polypeptide according to claim of 11, wherein the polypeptide can enhance transcription of transcriptional activators containing a proline-rich activation domain.

14. A purified polypeptide according to claim of 11, wherein the polypeptide can enhance transcription of transcriptional activators containing a glutamine-rich activation domain.

15. A purified polypeptide according to claim 11, wherein the polypeptide further associates with ASF/SF2 in vivo.

16. The purified polypeptide of claim 11, wherein the polypeptide comprises SEQ ID NO: 63.

17. The purified polypeptide of claim 16, wherein the polypeptide comprises SEQ ID NO: 5.

18. The purified polypeptide of claim 16, wherein the polypeptide comprises SEQ ID NO: 4.

19-20. (canceled)

21. A purified polypeptide having cotranscriptional activator activity, and which enhances ASF/SF2-mediated pre-mRNA splicing activity, the polypeptide comprising an amino acid sequence selected from the group consisting of:
a. the purified peptide of claim 3;
b. amino acid sequences that differ from that specified in (a) by one or more conservative amino acid substitutions, but which retain the cotranscriptional activator activity or ASF/SF2-mediated pre-mRNA splicing activity of the amino acid sequence shown in SEQ ID NO: 4; and
c. amino acid sequences having at least 75% sequence identity to the sequences specified in (a) or (b), but which retain the cotranscriptional activator activity of the amino acid sequence of SEQ ID NO: 4.

22. (canceled)

23. A purified polypeptide having cotranscriptional activator activity, and comprising an amino acid sequence selected from the group consisting of:
a. the purified peptide of claim 2;
b. amino acid sequences that differ from those specified in (a) by one or more conservative amino acid substitutions, but which retain the cotranscriptional activator activity of the amino acid sequence shown in SEQ ID NO: 2; and
c. amino acid sequences having at least 75% sequence identity to the sequences specified in (a) or (b), but which retain the cotranscriptional activator activity of the amino acid sequence shown in SEQ ID NO: 2.

24-35. (canceled)

36. A composition comprising a therapeutic amount of the polypeptide defined in of claim 19, and a pharmaceutically acceptable carrier.

37. A composition comprising a therapeutic amount of the polypeptide defined in claim 21, and a pharmaceutically acceptable carrier.

38. A composition comprising a therapeutic amount of the polypeptide defined in claim 23, and a pharmaceutically acceptable carrier.

39-70. (canceled)

71. The purified polypeptide of claim 21, wherein the peptide comprises at least 95% sequence identity to SEQ ID NO: 4 and retains cotranscriptional activator activity or ASF/SF2-mediated pre-mRNA splicing activity.

72. The purified polypeptide of claim 23, wherein the peptide comprises at least 95% sequence identity to SEQ ID NO: 2 and retains cotranscriptional activator activity.
